SEQUENCE LISTING (17 sequences)

SEQ ID NO: 1 - DNA, 1789 bp, artificial
  BPF3′5′ H#40.nt - viola sp
ggcacgaggacaacatggcaattctagtcaccgacttcgttgtcgcggctataattttcttgatcactcggttcttagttcgttctcttttcaagaaaccaacccgaccgctccccccgggtcctctcggttggcccttggtgggcgccctccctctcctaggcgccatgcctcacgtcgcactagccaaactcgctaagaagtatggtccgatcatgcacctaaaaatgggcacgtgcgacatggtggtcgcgtccacccccgagtcggctcgagccttcctcaaaacgctagacctcaacttctccaaccgcccacccaacgcgggcgcatcccacctagcgtacggcgcgcaggacttagtcttcgccaagtacggtccgaggtggaagactttaagaaaattgagcaacctccacatgctaggcgggaaggcgttggatgattgggcaaatgtgagggtcaccgagctaggccacatgcttaaagccatgtgcgaggcgagccggtgcggggagcccgtggtgctggccgagatgctcacgtacgccatggcgaacatgatcggtcaagtgatactcagccggcgcgtgttcgtgaccaaagggaccgagtctaacgagttcaaagacatggtggtcgagttgatgacgtccgccgggtacttcaacatcggtgacttcataccctcgatcgcttggatggatttgcaagggatcgagcgagggatgaagaagctgcacacgaagtttgatgtgttattgacgaagatggtgaaggagcatagagcgacgagtcatgagcgcaaagggaaggcagatttcctcgacgttctcttggaagaatgcgacaatacaaatggggagaagcttagtattaccaatatcaaagctgtccttttgaatctattcacggcgggcacggacacatcttcgagcataatcgaatgggcgttaacggagatgatcaagaatccgacgatcttaaaaaaggcgcaagaggagatggatcgagtcatcggtcgtgatcggaggctgctcgaatcggacatatcgagcctcccgtacctacaagccattgctaaagaaacgtatcgcaaacacccgtcgacgcctctcaacttgccgaggattgcgatccaagcatgtgaagttgatggctactacatccctaaggacgcgaggcttagcgtgaacatttgggcgatcggtcgggacccgaatgtttgggagaatccgttggagttcttgccggaaagattcttgtctgaagagaatgggaagatcaatcccggtgggaatgattttgagctgattccgtttggagccgggaggagaatttgtgcggggacaaggatgggaatggtccttgtaagttatattttgggcactttggtccattcttttgattggaaattaccaaatggtgtcgctgagcttaatatggatgaaagttttgggcttgcattgcaaaaggccgtgccgctctcggccttggtcagcccacggttggcctcaaacgcgtacgcaacctgagctaatgggctgggcctagttttgtgggccttaatttagagacttttgtgttttaaggtgtgtactttattaattgggtgcttaaatgtgtgttttaatttgtatttatggttaattatgactttattgtataattatttatttttcccttctgggtattttatccatttaatttttcttcagaattatgatcatagttatcagaataaaattgaaaataatgaatcggaaaaaaaaaaaaaaaaaaaaaaaaa

SEQ ID NO: 2 - protein, 506 aa, artificial
  BPF3′5′ H#40.aa - viola sp
MAILVTDFVVAAIIFLITRFLVRSLFKKPTRPLPPGPLGWPLVGALPLLGAMPHVALAKLAKKYGPIMHLKMGTCDMVVASTPESARAFLKTLDLNFSNRPPNAGASHLAYGAQDLVFAKYGPRWKTLRKLSNLHMLGGKALDDWANVRVTELGHMLKAMCEASRCGEPVVLAEMLTYAMANMIGQVILSRRVFVTKGTESNEFKDMVVELMTSAGYFNIGDFIPSIAWMDLQGIERGMKKLHTKFDVLLTKMVKEHRATSHERKGKADFLDVLLEECDNTNGEKLSITNIKAVLLNLFTAGTDTSSSIIEWALTEMIKNPTILKKAQEEMDRVIGRDRRLLESDISSLPYLQAIAKETYRKHPSTPLNLPRIAIQACEVDGYYIPKDARLSVNIWAIGRDPNVWENPLEFLPERFLSEENGKINPGGNDFELIPFGAGRRICAGTRMGMVLVSYILGTLVHSFDWKLPNGVAELNMDESFGLALQKAVPLSALVSPRLASNAYAT

SEQ ID NO: 3 - DNA, 4958 bp, artificial
  Pet gen DFR.nt - Petunia sp
gatctggggttgtcggcggtagaattggtcaaatcgatcctcggtggaagagcaggcggcgacatccttgagaagtgtggagattctggtggtacacgcacggcaccagtggcgaggtttaagccggcgccagtgtatgaagaggctgagcgccgacactgtggttagggggttttgggttgggtgtggtgggagtttgggggggtcgggttgtacgagagcatgtgatattgggggcagggggtgggagttggttttgggtgtgattggtggattagagtttatatttgtgttagaaaaaataatgttatggcttgtaaattgagaaatattgctaattttatgtaatataggagtgtaatttgtatatatgcgtaaaaaatccaagaaacatggtgttgcatcagggggaaaagcgagattcaagaagcttgataagatagataatccagaatatattcattagaggagctggaatgagaaggttagataggaaaatgcaaagtcttaacttttacccaatgtaaatgtagctatggtagttatggtagttggtagaagtagtagagtatcttggcccgagagtggaaaatgtccacctcagatagattgttgagctttattgtttacttggtaataaagttaattaattaccaaaaaaaaaaaggaagcatgtgattatttagtgcttatcactttattgaatgtctatgccatgtaaattttcctttatgttcatagcaagacaatattgatttaatacagattgacccacctacgtcgcaaatatactcaaatttaattttcaaacttacaagtatatgataataaagctttttaagtagtttatttgtgtaattgttccataaaaaaaatttgggcagacgtacttgtgggatttgccataaaaaagagaaatggaaatatttaaaaacctacgtaatcgacacccggctttggagaaaattctttagagaaatgtcgaaacctaacgaatgaagagttactggaaaatatgtcaaatatattttgacaaattcaaattaaaaggaagatgaactcaatcgttgcataaattattagattttttaatgaactttaaacttgattccctaacctgttgaacgtgttagggcttttgacctgaatttttaaactattaggactcctcttattgaagggatgaaaaagactcctaattgaaatatatctcctttatatgacttatcctttacttagaggagaagtaatagacaacaataaatagatgatcttcttctcacaatacacaacacaaattctacaatgtagtcttaggagaattttatttaggggagatttttcttcccatattatgtaggccagttggccaaactactttcaataacaacccttttgatatgtgtcattttcatatttgattcattgtcattaatgtttgtgtgttagcttccgcatgcatcatgttgttccgatcccaacaagtagtatcagagccatattcaactaatggttcgatgagccaggttgataaggttgaagatatgttcaaggcggtttcagagctgcaaccaatgacgataataaagttatataaaaaataggatggtaatgctacgtgtggagaaaagtttttcaaccatatattcaacaataatgttgctgctgcatctttaaaacaaaatactttttaacccatgttttggctacttttaaccaatctcagttttaactcatgcttattttaatgcttgggctcccttttaatccattcttgggctcatttttaacctgttgctgggcttctttgaaccaaaataatatttttaaacatgacaaacagcagtttgaagaccatgtgaagaaggaagatcaagattcttttgtccaaaattcaggccaaggcgggaattgttagtgtttttaccctgaatttttaacctattaggactactcttattgaagggatgaaaaagactcctaattagaatatatctcatttatatgacttatccttagaggagaagtaatagacaacaataaatagaggatcttcttctcacaacacacaacacaaattccacaatgtagtcttaggagaattttatttaggggagatttttcttcccatattatgtagcccagttggccaaactactttcaataacaacccttttgatatgtgtcatttttatatttgattcattgacattaatgtttgtgtgttagcttccgcatgcaccatgttgtttcgatcccaacggaagggacacatggtaacattcaatgccagtttctcaatttcgaccaacatccaaaagatgatattgcatatatggattgaaaatatgtttcttcatcacggtacgactcaatgatctttctaaaatcggaaaatttctaaggactgcatggttcgaaactcaaaaatgataaatatatccctttatcattctccactaaatattaggttgttcgaacctataaattacggctttccacacatcacgtgttgcgttacaactaaaccaaaaccattggaatcagtggcggagccacctttgggcaagggaattcaattgaaccctcttcaccgaaaatttgtactgcattgatattttaaattttgaacctcttattgaaaatcctgtctccgtcctgcttggagcaacaacacaactctatatgcatatgaaagagtgggtcctaagtaaccagatactacaccatccccacagccccattttcttctctctcagcaaccagtcctatttagttaatccaatgaagttactcaacgggccgttgagcacgtgctcaccatctaacattcccaatccttagacaacctacgtgcaagtactataaagacagatataaaccaacacataaataaagttcatcctgttgtaatttaactactagtaagtccactaaaattaacaaaatcttaagtccgactttccaacttccatatctgataatggcaagtgaagcagttcatgccccttcacctccggtggcagtgccgacagtttgcgtcactggagctgctggatttattggctcttggcttgtcatgagactccttgaacgcggttacaatgttcacgctactgttcgtgatcctggtatgttttgtttcgagagtttaacttctatgcattgctagcgtaaaagaactttgaaagtggtatgcgcgtgaagagaagtatgtgacattgataaaagtgtgccctttgtatggcatgcacttacgtaaagatgcatgattttgtagagaacaagaagaaggtgaaacatctgctggaactgccaaaggctgatacgaacttaacactgtggaaagcggacttgacagtagaaggaagctttgacgaggccattcaaggctgtcaaggagtatttcatgtagcaacacctatggatttcgagtccaaagaccctgaggtacgatcaaactagaagcaaatatacttgtggtcctttctacatttctggtctaaattctaacataactatgtaacatcgagatatgacagaatgaagtaatcaagccaacagtccggggaatgctaagcatcattgaatcatgtgctaaagcaaacacagtgaagaggctggttttcacttcatctgctggaactctcgatgtgcaagagcaacaaaaacttttctatgaccagaccagctggagcgacttggacttcatatatgctaagaagatgacaggatgggtttgtttggctattcttttcatttcgtaatacactctagtaacaaaaacagcattctcattgatacttgtgaattaatttcattgcagatgtattttgcttccaagatactggcagagaaagccgcaatggaagaagctaaaaagaagaacattgatttcattagcatcataccaccactggttgttggtccattcatcacacctacatttccccctagtttaatcactgccctttcactaattactggtatgctgtagtcttaaatattctacgtaattaaattgcacagatgatgtgcagttcttcctctcaccaaacaccccacaaattatttcaattaacaatatttttacagtcatgggtttaatcagattggggtatgcagggaatgaagctcattactgcatcattaaacaaggtcaatatgtgcatttggatgatctttgtgaggctcacatattcctgtatgagcaccccaaggcagatggaagattcatttgctcgtcccaccatgctatcatctacgatgtggctaagatggtccgagagaaatggccagagtactatgttcctactgagtaagcctctctcttctgtattcccaagtatagtaggctccttcattgagtgatggcttagtaactcactcgtgggtaaataacaggtttaaagggatcgataaagacctgccagtggtgtctttttcatcaaagaagctgacagatatgggttttcagttcaagtacactttggaggatatgtataaaggggccatcgatacttgtcgacaaaagcagctgcttcccttttctacccgaagtgctgaagacaatggacataaccgagaagccattgccatttctgctcaaaactatgcaagtggcaaagagaatgcaccagttgcaaatcatacagaaatgttaagcaatgttgaagtctagaactgcaatcttacaagataaagaaagcttgccaagcaatatgtttgctactaagttctttgtcatctgtttgagggttttcaaaactaaatcagtaaatttttcgatgcatatagagaagttcttgtcttgctaaattacgggcagctaaacaataggatatcaagaatcccgtgctatatttttcaggaaaataaaatctataatcatttcagggaatctggatactaatacaaggacgtattttccaatttataagctttgcaaaagcaagatc

SEQ ID NO: 4 - protein, 380 aa, artificial
  Pet gen DFR.aa - Petunia sp
MASEAVHAPSPPVAVPTVCVTGAAGFIGSWLVMRLLERGYNVHATVRDPENKKKVKHLLELPKADTNLTLWKADLTVEGSFDEAIQGCQGVFHVATPMDFESKDPENEVIKPTVRGMLSIIESCAKANTVKRLVFTSSAGTLDVQEQQKLFYDQTSWSDLDFIYAKKMTGWMYFVSKILAEKSAMEETKKKNIDFISIIPPLVVGPFITPTFPPSLITALSLITGNEAHYCIIKQGQYVHLDDLCEAHIFLYEHPKADGRFICSSHHAIIYDVAKMVREKWPEYYVPTEFKGIDKDLPVVSFSSKKLTDMGFQFKYTLEDMYKGAIETCRQKQLLPFSTRSAADNGHNREAIAISAQNYASGKENAPVANHTEMLTNVEV

SEQ ID NO: 5 - DNA, 37 bp, artificial
  DFRint3 5S F
gcatctcgagggatcctcgtgatcctggtatgttttg

SEQ ID NO: 6 - DNA, 37 bp, artificial
  DFRint3 5S R
gcattctagaagatctcttcttgttctctacaaaatc

SEQ ID NO: 7 - DNA, 37 bp, artificial
  ds carnDFR F
gcattctagactcgagcgagaatgagatgataaaacc

SEQ ID NO: 8 - DNA, 35 bp, artificial
  ds carnDFR R
gcatagatctggatccgagattgttttctgctgcg

SEQ ID NO: 9 - DNA, 1215 bp, artificial
  Carn DFR.nt - Dianthus caryophyllus
ctcgaatttgatttgatacacacttacaaaatacaatacatttgtaacaaaaaaagatggtttctagtacaattaacgagacactagacggtaaacatgacattaataaggttgggcaaggcgagaccgtctgcgtgaccggtgcatccggtttcattggttcatggctcatcatgcgactccttgagcgtggctacaccgttcgagccacagttcgtgatcccgataacactaaaaaagtgcaacatttgttggatttgccaaatgccaagactaacttgacactttggaaagcggacctacacgaagaaggcagctttgatgcagccgttgatggatgcaccggcgtgtttcatatcgccacacctatggactttgagtccaaggatcccgagaatgagatgataaaacctacaataaatggaatgttggacatactaaagtcatgtgtgaaggccaaactaaggagggtggtttttacgtcatctggtggaactgtgaatgtcgaagcgactcaaaaacccgtctatgacgagacttgttggagtgctctcgacttcattcgctctgttaagatgactggatggatgtacttcgtgtcaaaaatactagcagagcaagcagcgtggaagtacgcagcagaaaacaatctcgaattcatcagtatcattccacccctcgttgttgggccctttatcatgccttctatgcctcctagcctcattaccgcgttatcccccataacaagaactgaatcacattatacaataataaagcaaggacaattcgtgcacttggatgacctttgtatgtctcacatcttcttatacgagaatccgaaggcaaatggtcgatatattgcctcagcctgtgctgctaccatttacgacatcgcaaagatgctcagggaagaataccctgagtacaatgtccccaccaaattcaaggactacaaggaggacatggggcaagtacaattctcgtcgaagaaactgacggatctcgggtttgagttcaaatacgggttgaaggacatgtacacagcagctgtcgagtcttgcagagcaaaagggcttcttcctctttccctagaacatcatctttgtgttttccgagtgacgcttatattttttaaataaatcagtcctgatgtaacgatgcacgttttcgataagtgatttataaatttaatttcgtgtcgaaaaaaaaaaaaaa

SEQ ID NO: 10 - protein, 360 aa, artificial
  Carn DFR.aa - dianthus caryophyllus
MVSSTINETLDGKHDINKVGQGETVCVTGASGFIGSWLIMRLLERGYTVRATVRDPDNTKKVQHLLDLPNAKTNLTLWKADLHEEGSFDAAVDGCTGVFHIATPMDFESKDPENEMIKPTINGMLDILKSCVKAKLRRVVFTSSGGTVNVEATQKPVYDETCWSALDFIRSVKMTGWMYFVSKILAEQAAWKYAAENNLEFISIIPPLVVGPFIMPSMPPSLITALSPITRTESHYTIIKQGQFVHLDDLCMSHIFLYENPKANGRYIASACAATIYDIAKMLREEYPEYNVPTKFKDYKEDMGQVQFSSKKLTDLGFEFKYGLKDMYTAAVESCRAKGLLPLSLEHHLCVFRVTLIFFK

SEQ ID NO: 11 - DNA, 1006 bp, artificial
  ThMT.nt
ttcaattccgccattttctccaataataacattcataaatacaatcagcagcagcaaaaatgaaagataagttctatggcaccattttgcagagcgaagccctcgcaaagtatctgttagagacaagtgcctatccacgagaacatccgcagctcaaagaactaaggagcgcaactgtggacaagtatcaatattggagcttgatgaatgttccagctgatgaggggcagttcatttcaatgttactgaaaattatgaacgcaaaaaagacaattgaagttggagttttcacaggctactcactcctatcaactgctctggctctacctgatgatggcaaaatcgttgccattgatcctgatagagaagcttatgagactggtttgccatttatcaagaaagcaaacgtggctcataaaatccaatacatacaatctgatgccatgaaagtcatgaatgacctcattgctgccaagggagaagaagaagaggggagctttgactttgggttcgtggatgcagacaaagaaaactacataaactaccacgagaaactgttgaagctggttaaggttggagggatcataggatacgacaacactctgtggtctggaacagttgctgcatctgaagacgatgagaataatatgcgagactacttaagaggttgcagagggcatatcctcaaactaaactcctttctcgcaaacgatgatcggattgaattggctcacctctctattggagatggactcaccttgtgcaaacgtctcaaataataattttcaactttattattattgtttcataaaaagcatttactgctggcctggcctggcctgtttcagcatcttatatttctattgttctaaatattttagttatcttgtttatcaacttgtctgtcttatatgtttaaaagaaagatgtcatgtaattgtaactcgatcgggctcttgtaatattataatgaattttattgattttcaaaaaaaaaaaaaaaaaa

SEQ ID NO: 12 - protein, 239 aa, artificial
  ThMT.aa
MKDKFYGTILQSEALAKYLLETSAYPREHPQLKELRSATVDKYQYWSLMNVPADEGQFISMLLKIMNAKKTIEVGVFTGYSLLSTALALPDDGKIVAIDPDREAYETGLPFIKKANVAHKIQYIQSDAMKVMNDLIAAKGEEEEGSFDFGFVDADKENYINYHEKLLKLVKVGGIIGYDNTLWSGTVAASEDDENNMRDYLRGCRGHILKLNSFLANDDRIELAHLSIGDGLTLCKRLK

SEQ ID NO: 13 - DNA, 1745 bp, artificial
  ThFNS.nt
ggaattcggcacgagatcgaaaccgctatatcattacatttacaacagcgctaaaaaaatatatataaagcatggacacagtcttaatcacactctacaccgccctgttcgtcatcaccaccaccttcctcctcctcctccgccgaaggggaccaccgtctccgcccggtcctctctccctacccataattggccacctccacctcctcggcccaagactccaccacacgttccatgaattctcactcaaatacggcccattgatccagctcaagctcggctcgatcccgtgcgtcgtggcctcgacgcccgagctcgcgagagagtttcttaagacgaacgagctcgcgttctcctctcgcaagcactctacggccatagacatcgtcacctacgactcgtcctttgctttctctccgtacggaccctactggaagtacatcaagaaactgtgtacctacgagctgctcggagcgaggaacctcggacactttcagcccattaggaatctcgaggtcaggtcctttctgcagcttctgatgcacaagagctttaagggcgagagtgtgaatgtgacagacgagctggtgaggctgacgagcaatgtgatatcccacatgatgctgagcataaggtgctcggaagatgaaggcgatgctgaggcggcgagaacagtgatacgcgaggtgacgcagatatttggggaattcgatgttacggacataatatggttttgcaagaaattcgatctgcaggggataaagaagaggtcagaggatattcagaggaggtatgatgctttgctcgagaagattattagtgatagagagagatcgaggaggcaaaatcgtgataagcatggtggcggtaacaatgaggaggccaaggattttcttgatatgttgcttgatgtgatggagagtggggacacggaggtcaaattcactagagagcatctcaaggctttgattctggatttcttcacggccggtacggacacaacagccatagccaccgagtgggccatcgccgagctcatcaacaacccgaacgtcttgaagaaggcccaagaagaaatatcccggatcatcggaaccaagcggatcgtacaagaatccgacgccccagacctaccctacctccaggccatcatcaaggagacgttccggctccacccaccgatcccgatgctctcgcgtaagtccacctccgattgcacggtcaacggctacaaaatccaagccaagagcctcttgttcgtgaacatatggtccatcggtcgaaaccctaattactgggaaagccctatggagttcaggcccgagcggttcttggagaagggacgcgagtccatcgacgtcaagggccagcactttgagctcttgccttttgggacgggccgcaggggctgtcccggtatgttgctggctatacaagaggtggtcagcatcattgggaccatggttcagtgcttcgactggaaattggcagatggttcgggcaataatgtggacatgaccgaacggtctggattgaccgctccgagagcgttcgatctggtttgccggttgtatccacgggttgacccggccacaatatcgggtgcttgatgtagtagggtgaggcgcgtgttggtgttttatctttcggttttgttctgttagtattattatggtctgtgttgaagcctcaaggattttaaaaaaaaaaaaaaaaaaaaaaaaaaaaaaaaaaaaaaaaaaaaa

SEQ ID NO: 14 - protein, 511 aa, artificial
  ThFNS.aa
MDTVLITLYTALFVITTTFLLLLRRRGPPSPPGPLSLPIIGHLHLLGPRLHHTFHEFSLKYGPLIQLKLGSIPCVVASTPELAREFLKTNELAFSSRKHSTAIDIVTYDSSFAFSPYGPYWKYIKKLCTYELLGARNLGHFQPIRNLEVRSFLQLLMHKSFKGESVNVTDELVRLTSNVISHMMLSIRCSEDEGDAEAARTVIREVTQIFGEFDVTDIIWFCKKFDLQGIKKRSEDIQRRYDALLEKIISDRERSRRQNRDKHGGGNNEEAKDFLDMLLDVMESGDTEVKFTREHLKALILDFFTAGTDTTAIATEWAIAELINNPNVLKKAQEEISRIIGTKRIVQESDAPDLPYLQAIIKETFRLHPPIPMLSRKSTSDCTVNGYKIQAKSLLFVNIWSIGRNPNYWESPMEFRPERFLEKGRESIDVKGQHFELLPFGTGRRGCPGMLLAIQEVVSIIGTMVQCFDWKLADGSGNNVDMTERSGLTAPRAFDLVCRLYPRVDPATISG

SEQ ID NO: 15 - DNA, 2544 bp, artificial
  carnANS 5′
actcgaaatgatactgtgagtctgcgataggctcgttttgaggcggaaattatgattttacgacgtgatcaattgagcatgacttaaatttgcgtcttctcagtcgtcgttgcattgcaatttttagtattttcaggtgctctgaaagtgtttagtacatcgtttttaaaatggatatctttttgttctggtcgacttactcttcgctttttaatgcagacgtgcccgttattgctacgtgtattcacaaaggtatgaccgtgttctgtagcgtctaatgataatatatgaagtcgaggttgcatttgtactagtccgataataattagtatcgttttcatactgatactagatcggaggtcaccatacccgtgaagatttttctgtgagaggaaaagaacccaaggacgaggttcaaatctacacatggaaagacgccacgcttcgtgagttaactgaccttgtatgtcgccatcgcttagcgtagcgctgaacatcgttttcaccctgctccatccatcaaccatttattggtcttatacatgtgtgattgcgttgttcttacatttaggtgaaagacgtttctccagctgctaggagtcgagatgcgaaattgtcgtttgcgactgtataccttgatagaaatggatgcatgcaagtaaagaaggtatcttctaattcatctttcgtagagacatagcgtgaatttggacggggtctttggtttgagaaagataacagctttacgtatttttgtagatgggtgaaaccttttcaaatccgtataagcgtaaagacgacaactgggctttaggggacacattctttcaggtataattgatgcgactaacaatagtctccactgatcatattctactcttctacgttcgatactgactgtttctggttatttggtagacaggagattatttggacgtagcaattcagtagcgtagagatgtttccacacgtgttatcgtaaaagaagcaagataagcctaatgcctagggtggtggtatgacttccgttgcttatcgatcgtgcttgtaagtaatttccgtcttatcttttcctgttatataaagttaatcttctctaggactttcatgaaccttgtttgtgtatttatttctcgatcaacatgatagagctagtttttaagcaacgtatactagtagtctattggaagttaagacacggttcttaaaaaggtacgatccaagtgaagcatgttagatatgacactttcttctagggacgactctcgtatgccacccgactttttcaattttttttgtgaatgttagatgtgtgtatataatgcatccgaaagatgtctcaacgaacaaatgagccacctacttcgatcactcgctatcaatgttattaatgccttgttgattttaatagttgatcaataatagtaaaatctattcaagggtatagtctcccgttcacactcatcggggttacactagcgagctccattaatcggtgccttaatcgagacgctaagaactataccatgacctagtcagcgccatgggactgatgtaggccacacaatctcgatgatccgaaaacgctagagttcaagacctagttcgagaccatggtcacggtttcaaccgcgatatctcaacaatgcaatttttttcgagactagacagacgaataagtcttgtgtacgatgggtagctagtgaattaaaggtaatcactttactcgtgttcacaagaagaccattcatatgaacatttcgtgttctcagacgagctcctttcgctagtttggtaaacttgtttggtgttacctggcttatcatagcccactcaatttggcgaaaacataacaattgtttcacatgcgaaggtctacacacccatactcgtgataaaaacggttgcatttattattattgcctttcgagcaatttacgttgtttgaagtttgtgtaaaaacaaaatcgatctataattactgactggaacggtaatatcaaaactgttaggacctgttcttttcggcttataagagctgaacttataaaagctgtacttataggagctgagctgaactgaacttataagagttgaactgaacttataagggctgaaattaagctgtaaagaacaggttcttatttcgcctgatttgaatccgactaaagttggatgaagatagaggcgagaaacgctcccctttccacagtttgactctactcgaatccgaccaaaattggatgtaaattagaagtgagcattcctcatgccaaccaccgttcttggtcgtgaaaacatgatttgttagtctgctgtatacttcccaaacccgtgataatctgccacacttccaacacttaattgatttttatcaaaattcctacagttttttggttttactcctaaactatgctgtctttttacaagttgttacacctttgtcaacaactttatgctttattatatttcttatataaagaccatataacttccctacactaatgccacaacacttaaaagcatacaacacaaacctcataa

SEQ ID NO: 16 - DNA, 822 bp, artificial
  carnANS 3′
actcactacgtgtttacgattgtgtgtttgtcgtgtttgcttaaatcgaccatgtcctcaactttgagaaatcaatagttgtacttttgagttattgttatctcaataacgatattattgcgttatacggagtactgtttagaagacgatatgtaataaatgaaatcgtagttgtctatagcttcatgattatcgttacactattattcttatacttcatctttattttagttaatcttatcaaatacttcgtatattggaaaatttcaaaaagttacttaaaaaataataaaatacaccgaatttcagaaaactcaggaatattcaggcgtaataacatccgttaataccgaaaatattatgaaacgtggcaaatgttggacggtgtgcaatatgagacacaaaaaaacaattgtgaaaaatctttggtcgtaacgaattgcgtcctaaatcatcataataatacaaataaaaaaatgtcattttcattaaatttcttctcttctgtaatcatagaattttattccaacttcatcatctaacttcaggtacatttctccttctctaacttattatcattactttttattgttttaaattatattttttatgtctattaaacgatttaaaatgatcacagtttactctgtacttgtcgtaaataatgtgctgtatgcatacaacagtcttattggtttacgtgaactgttagctgtggttgcatacaatttagattgaaccgacagatatcgcctttactgtcggagggagctgttacagacttacgatactgttcgtaaatggcaagcttgatatcgaatt

SEQ ID NO: 17 - DNA, 2934 bp, artificial
  RoseCHS 5′
aagcttcagcaagagttgaagaaatagggacagagccatccatgtgctttgatgaatctgatgggatacaaaatgtgaaagattcacttgctgatttatccagaatttcttcatatagtgaggagaatgttgaaagatctaatgatgagcactctgttaaactagacggaattcatgtgcagcacgagtgtcatgagggcagtgaagaagacaaacctgatggtaagagcggtgagaatgcagttgatctggctaatcatggcatggctcgaactgatttttgtcagataacagaagagattgagaatggagtagtcatcactgagatgagcaacattgccaaccctgataaaactgatattccaaacggggtgcctcaaaatgagactgatgatggatttaataacactcaggatgatgctaatacaaaggaagtgacagaagagaattctgacagacgtgcgaaggaagtgacagaagagaattctgacaaagatgttttgaagaatatccttgaattctcacgtgcttcttctgtggtggattttgaaattccagtgttggatgtgaaatttacttctcttgaaagttgcagtgccacttgttctcttgcagcccttttgtctgaatcgccggaatcaatgactgaagcaccttgtgtgaggcaaattgatgatgtgcccccggttggtgaggagtctagcttgattttggtggaagatcgggagccggttggtcctactcctgatggtaatttttctgtggatatggattactatagtgtagcagaacctttgagcacatgggatgcgaatctgcagtgtgaaacatcaaatagccatgagacttttgctgcaagtctcatttgatagcttctgtgttaataactttgttagtctgtacataaatttgtctagacaagaattggtcgtgtactatcgtgtgtttttgccgtgctttagtactcatgaaccaattcagagaaaactggctgcatattttgaggagtctctgaattcttcaatgctcaactggtatgcatgtaggtggcatatcacttcagggattcttctattctttaactttacgcatcttgacattttgtatataacaaaatcaggtctattgggtgaaagtaattggctagaatggaaagctctacggttttaccgcaggtcaattttcatagctccacaagtgaattgaaaatgctcataggctttatgtttgtcctccacctctggcgacgatgtttgttggggagttaactcaaacctaccaccaaactcgaacccatcttccataatttataatacaaatttgcgatcatttgttcatccaattattgtgacactcggctaccacccaaaatatcggtcacagacccaaacgtattgtcacaacaaatcgtgtctctcgcattaaacacagctagaaagaagagttgaacccacaattcgagcacccactacctatgtacgaagtcatgagttcgagtcaccataggggtagaagtgaaatcatttgatcatctttaaagaaataaaaggaagagttgaacccacaattggctcttgtcccaaaaagaactaatagttcagtgcaccgacgtgtatttgcaccgacataaatggattgttagattatattaaatacactcttaggttattaataaaaatattaattataaatatcaaaagttgagatcatcttataaatgttgggtcagttacaccgtcggtgcatagaataatttccaaactatataatagccttcattttctgatttagctcatgggacatgattgctataaataattgtactcgtagaggcatacttgtgtctttttatacagttgtactgaagctcagaaaagtttatgaaggtgagaactgagaagggcaaggcatttggtagttgaggtatatgagagcatgaaccccatgcattgcagctaccacctctcttttttccttcttcccatacaaataaaaccaactcttctcacctaagtctatcatctttatttatggcagctcttgcttaattagctcatctatattatattatttatctataatatgtgtcactctgtctacctaccagcccaaaataaaactgataatagtcaatttgatgatattttttgttttttgttttgttttgtcttttttgtattgatttttttaaaattaaaatgacttcattttttgtttttgtttttttttctatttttttttatagaaaaattggcaaactttcattatctgttattgatgacaattaagccattaaaacctataattaattatctttcaattcgagtaaatttaaaacggtgtaaaattaaaatatgatcgtattcttaaatgaataaaactcacttaataatagtaatacttgaatcacatctacgaacatagattcttttcatccagtctaaccatgtttgaatatatagagtttgattatggttatgtctttgtccacattttggtttgtaaataaatgtgcaacggaggtatggtactgttgctctatcaaattcaagtttgaattaaaagaaaaaaaaaaagacgatattttgtgcgctttgtttggtaggtaaaacgagagaacaaacgcattccaaatcatgcggattttgatcggcaacacacaccacaaaaaaccgtacacgatgcacgtgccatttgccgggggtttctaacaaggtaattgggcaggcacgtgatcccccagctacccacctctcgcttcccttctcaaactccttttccatgtatatatacaaccccttttctcagaccattatattctaacatttttgctttgctattgtaacgcaacaaaaactgctcattccatccttgttcctccccattttgatcttctctcgacccttctccgagatgggtaccgagctcgaattc

(19) United States
(12) Patent Application Publication
(10) Pub. No.: US 2011/0321184 A1
(43) Pub. Date: Dec. 29, 2011

(54) PLANT WITH ALTERED INFLORESCENCE

(76) Inventor: Filippa Brugliera, Victoria (AU)

(21) Appl. No.: 13/140,389

(22) PCT Filed: Dec. 18, 2009
(86) PCT No.: PCT/AU09/01659

§ 371 Date: Sep. 12, 2011

(30) Foreign Application Priority Data
Feb. 19, 2008 (US) 61139354

(51) Int. Cl. A01H 5/02; A01H 1/02; C12N 15/82; A01H 5/00; C12N 5/10

(52) U.S. Cl. 800/260; 800/298; 800/323.3; 435/419; 800/278

(57) ABSTRACT

The invention relates to genetically engineered plants with altered inflorescence. Plants such as spray carnations are transformed with a non-indigenous flavonoid 3′,5′ hydroxylase (F3′5′H) and dihydroflavanol-4-reductase (DFR) in conjunction with a genetic suppressor of indigenous DFR. Preferably the substrate specificity of the indigenous DFR is different to the non-indigenous DFR in order to enhance the colour of the inflorescence.

### FILING DATA

This application is associated with and claims priority from U.S. Provisional Patent Application No. 61/139,354, filed on 19 Dec. 2008, entitled “A Plant”, the entire contents of which, are incorporated herein by reference.

### FIELD

The present invention relates generally to the field of genetic modification of plants. More particularly, the present invention is directed to genetically modified plants expressing desired color phenotypes.

### BACKGROUND

Bibliographic details of the publications referred to by the author in this specification are collected at the end of the description.

Reference to any prior art in this specification is not, and should not be taken as, an acknowledgment or any form of suggestion that this prior art forms part of the common general knowledge in any country.

The flower or ornamental or horticultural plant industry strives to develop new and different varieties of flowers and/or plants. An effective way to create such novel varieties is through the manipulation of flower color. Classical breeding techniques have been used with some success to produce a wide range of colors for almost all of the commercial varieties of flowers and/or plants available today. This approach has been limited, however, by the constraints of a particular species' gene pool and for this reason it is rare for a single species to have the full spectrum of colored varieties. For example, the development of novel colored varieties of plants or plant parts such as flowers, foliage, fruits and stems would offer a significant opportunity in both the cut flower, ornamental and horticultural markets. In the flower or ornamental or horticultural plant industry, the development of novel colored varieties of carnation is of particular interest. This includes not only different colored flowers but also anthers and styles.

Flower color is predominantly due to three types of pigment: flavonoids, carotenoids and betalains. Of the three, the flavonoids are the most common and contribute a range of colors from yellow to red to blue. The flavonoid molecules that make the major contribution to flower color are the anthocyanins, which are glycosylated derivatives of cyanidin and its methylated derivative peonidin, delphinidin and its methylated derivatives petunidin and malvidin and pelargonidin. Anthocyanins are localized in the vacuole of the epidermal cells of petals or the vacuole of the sub epidermal cells of leaves.

The flavonoid pigments are secondary metabolites of the phenylpropanoid pathway. The biosynthetic pathway for the flavonoid pigments (flavonoid pathway) is well established (Holton and Cornish, Plant Cell 7:1071-1083, 1995; Mol et al, Trends Plant Sci. 3:212-217, 1998; Winkel-Shirley, Plant Physiol. 126:485-493, 2001a; and Winkel-Shirley, Plant Physiol. 127:1399-1404, 2001b, Tanaka and Mason, In Plant Genetic Engineering, Singh and Jaiwal (eds) SciTech Publishing Llc., USA, 1:361-385, 2003, Tanaka et al, Plant Cell, Tissue and Organ Culture 80:1-24, 2005, Tanaka and Brugliera, In Flowering and Its Manipulation, Annual Plant Reviews Ainsworth (ed), Blackwell Publishing, UK, 20:201-239, 2006) and is shown in FIG. 1. Three reactions and enzymes are involved in the conversion of phenylalanine to p-coumaroyl-CoA, one of the first key substrates in the flavonoid pathway. The enzymes are phenylalanine ammonia-lyase (PAL), cinnamate 4-hydroxylase (C4H) and 4-coumarate: CoA ligase (4CL). The first committed step in the pathway involves the condensation of three molecules of malonyl-CoA (provided by the action of acetyl CoA carboxylase (ACC) on acetyl CoA and CO₂) with one molecule of p-coumaroyl-CoA. This reaction is catalyzed by the enzyme chalcone synthase (CHS). The product of this reaction, 2′,4,4′,6′, tetrahydroxy-chalcone, is normally rapidly isomerized by the enzyme chalcone flavanone isomerase (CHI) to produce naringenin. Naringenin is subsequently hydroxylated at the 3 position of the central ring by flavanone 3-hydroxylase (F3H) to produce dihydrokaempferol (DHK).

The pattern of hydroxylation of the B-ring of DHK plays a key role in determining petal color. The B-ring can be hydroxylated at either the 3′, or both the 3′ and 5′ positions, to produce dihydroquercetin (DHQ) or dihydromyricetin (DHM), respectively. Two key enzymes involved in this part of the pathway are the flavonoid 3′ hydroxylase (F3′H) and flavonoid 3′,5′ hydroxylase (F3′5′H), both members of the cytochrome P450 class of enzymes.

F3′H is a key enzyme in the flavonoid pathway leading to the cyanidin-based pigments which, in many plant species contribute to red and pink flower color. F3′5′H leads to the production of delphinidin based anthocyanins which, in many species contribute to the purple, violet and blue flower colors.

Nucleotide sequences encoding F3′5′Hs have been cloned (see International Patent Application No. PCT/AU92/00334 incorporated herein by reference and Holton et al, Nature, 366:276-279, 1993 and International Patent Application No. PCT/AU03/01111 incorporated herein by reference). These sequences were efficient in modulating 3′,5′ hydroxylation of flavonoids in petunia (see International Patent Application No. PCT/AU92/00334 and Holton et al, 1993 supra), tobacco (see International Patent Application No. PCT/AU92/00334), carnations (see International Patent Application No. PCT/AU96/00296 incorporated herein by reference) and roses (see International Patent Application No. PCT/AU03/01111).

The production of the colored anthocyanins from the dihydroflavonols (DHK, DHQ, DHM), involves dihydroflavonol-4-reductase (DFR) leading to the production of the leucoanthocyanidins. The leucoanthocyanidins are subsequently converted to the anthocyanidins, pelargonidin, cyanidin and delphinidin. These flavonoid molecules are unstable under normal physiological conditions and glycosylation at the 3-position, through the action of glycosyltransferases, stabilizes the anthocyanidin molecule thus allowing accumulation of the anthocyanins. In general, the glycosyltransferases transfer the sugar moieties from UDP sugars to the flavonoid molecules and show high specificities for the position of glycosylation and relatively low specificities for the acceptor substrates (Seitz and Hinderer, Anthocyanins. In: Cell Culture and Somatic Cell Genetics of Plants. Constabel and Vasil (eds.), Academic Press, New York, USA, 5:49-76, 1988). Anthocyanins can occur as 3-monosides, 3-biosides and 3-triosides as well as 3,5-diglycosides and 3,7-diglycosides associated with the sugars glucose, galactose, rhamnose, arabinose and xylose (Strack and Wray, In: The Flavonoids—Advances in Research since 1986. Harborne, J. B. (ed), Chapman and Hall, London, UK, 1-22, 1993).

Glycosyltransferases involved in the stabilization of the anthocyanidin molecule include UDP glucose: flavonoid 3-glucosyltransferase (3GT), which transfers a glucose moiety from UDP glucose to the 3-O-position of the anthocyanidin molecule to produce anthocyanidin 3-O-glucoside.

Many anthocyanidin glycosides exist in the form of acylated derivatives. The acyl groups that modify the anthocyanidin glycosides can be divided into two major classes based upon their structure. The aliphatic acyl groups include malonic acid or succinic acid and the aromatic class includes the hydroxy cinnamic acids such as p-coumaric acid, caffeic acid and ferulic acid and the benzoic acids such as p-hydroxybenzoic acid. For example in carnation the anthocyanins exist as malylated anthocyanins (Nakayama et al, Phytochemistry, 55, 937-939, 2000; Fukui et al, Phytochemistry, 63(1):15-23, 2003).

In addition to the above modifications, pH of the vacuole or compartment where pigments are localized and co-pigmentation with other flavonoids such as flavonols and flavones can affect petal color. Flavonols and flavones can also be aromatically acylated (Brouillard and Dangles, In: The Flavonoids—Advances in Research since 1986. Harborne, J. B. (ed), Chapman and Hall, London, UK, 1-22, 1993).

Carnation flowers can produce two types of anthocyanidins, depending on their genotype-pelargonidin and cyanidin. In the absence of F3′H activity, anthocyanins derived from pelargonidin are produced otherwise those derived from cyanidin are produced. Pelargonidin derived pigments are usually accompanied by kaempferol, a colorless flavonol. Cyanidin derived pigments are usually accompanied by both kaempferol and quercetin. Both pelargonidin and kaempferol are derived from DHK; both cyanidin and quercetin are derived from DHQ (FIG. 1).

The substrate specificity shown by DFR regulates the anthocyanins that a plant accumulates. Petunia and cymbidium DFRs do not reduce DHK and thus they do not accumulate pelargonidin-based pigments (Forkmann and Ruhnau, Z Naturforsch C. 42c, 1146-1148, 1987, Johnson et al, Plant Journal, 19, 81-85, 1999). Many important floricultural species including iris, delphinium, cyclamen, gentian, cymbidium, nierembergia are presumed not to accumulate pelargonidin derived pigments due to the substrate specificity of their endogenous DFRs (Tanaka and Brugliera, 2006 supra).

In carnation, the DFR enzyme is capable of metabolizing DHK to leucopelargonidin, the precursor to pelargonidin-based pigments, giving rise to apricot to brick-red colored carnations and DHQ to leucocyanidin, the precursor to cyanidin-based pigments, producing pink to red carnations. Carnation DFR is also capable of converting DHM to leucodelphinidin (Forkmann and Ruhnau, 1987 supra), the precursor to delphinidin-based pigments. Wild-type or classically-derived carnation lines do not contain a F3′5′H enzyme and therefore do not synthesize DHM.

The petunia DFR enzyme has a different specificity to that of the carnation DFR. It is able to convert DHQ through to leucocyanidin, but it is not able to convert DHK to leucopelargonidin (Forkmann and Ruhnau, 1987 supra). It is also known that in petunia lines containing the F3′5′H enzyme, the petunia DFR enzyme can convert the DHM produced by this enzyme to leucodelphinidin which is further modified giving rise to delphinidin-based pigments which are predominantly responsible for blue colored flowers (see FIG. 1). Even though the petunia DFR is capable of converting both DHQ and DHM, it is able to convert DHM far more efficiently, thus favoring the production of delphinidin (Forkmann and Ruhnau, 1987 supra).

Carnations are one of the most extensively grown cut flowers in the world.

There are thousands of current and past cut-flower varieties of cultivated carnation. These are divided into three general groups based on plant form, flower size and flower type. The three flower types are standards, sprays and midis. Most of the carnations sold fall into two main groups—the standards and the sprays. Standard carnations are intended for cultivation under conditions in which a single large flower is required per stem. Side shoots and buds are removed (a process called disbudding) to increase the size of the terminal flower. Sprays and/or miniatures are intended for cultivation to give a large number of smaller flowers per stem. Only the central flower is removed, allowing the laterals to form a ‘fan’ of stems.

Spray carnation varieties are popular in the floral trade, as the multiple flower buds on a single stem are well suited to various types of flower arrangements and provide bulk to bouquets used in the mass market segment of the industry.

Standard and spray cultivars dominate the carnation cut-flower industry, with approximately equal numbers sold of each type in the USA. In Japan, Spray-type varieties account for 70% of carnation flowers sold by volume, whilst in Europe spray-type carnations account for approximately 50% of carnation flowers traded through out the Dutch auctions. The Dutch auction trade is a good indication of consumption across Europe.

Whilst standard and midi-type carnations have been successfully manipulated genetically to introduce new colors (Tanaka and Brugliera, 2006 supra; see also International Patent Application No. PCT/AU96/00296), this has not been applied to spray carnations. There has been absence of blue color in color-assortment in carnation, only recently filled through the introduction of genetically-modified standard-type carnation varieties. However, standard-type varieties can not be used for certain purposes, such as bouquets and flower arrangements where a large number of smaller carnation flowers are needed, such as hand-held arrangements, and small table settings.

One particular spray carnation which is particularly commercially popular is the Cerise Westpearl line of carnations (Dianthus caryophyllus cv. Cerise Westpearl). The variety has excellent growing characteristics and a moderate to good resistance to fungal pathogens such as Fusarium. Cerise Westpearl is a sport of Westpearl. However, before the advent of the present invention, purple/blue spray carnations were not available.

White Unesco is a classically-derived carnation of the midi-type. It is white and does not normally produce anthocyanins primarily because the petals do not accumulate carnation DFR transcripts and so when White Unesco was transformed with Viola F3′5′H and a petunia DFR gene, over 80% of the anthocyanins produced were delphinidin based (see International Patent Application PCT/AU96/00296). Although this process has been useful in obtaining carnation lines with a purple/violet petals, it is limited to the identification of white lines that are mutant in the ability to accumulate petal carnation DFR mRNA or functional DFR enzymes in the petals but have the rest of the anthocyanin pathway intact so that the DHM produced can be converted to stable, colored anthocyanins. Of the 13 lines analyzed (see International Patent Application PCT/AU96/00296), only two were deficient in carnation DFR but intact in the ability to produce anthocyanins. Of the two, only one (White Uncesco) resulted in the production of purple/violet petals upon the introduction of F3′5′H and a petunia DFR.

The application of a similar approach using Viola F3′5′H and a petunia DFR transformed into a colored line such as Cerise Westpearl has not yielded significant novel colored products.

There is a need, therefore, to find an alternative means of producing novel colored purple/mauve flowers using colored lines such as Cerise Westpearl.

### SUMMARY

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO). The SEQ ID NOs correspond numerically to the sequence identifiers <400>1 (SEQ ID NO:1), <400>2 (SEQ ID NO:2), etc.

A summary of sequence identifiers used throughout the subject specification is provided in Table 1.

The present invention provides genetically modified plants exhibiting altered inflorescence. More particularly, the present invention provides genetically modified carnations and even more particularly genetically modified carnation sprays exhibiting altered inflorescence. The altered inflorescence is a color in the range of red-purple to blue such as purple and mauve to blue color in the tissue or organelles including flowers, petals, anthers and styles. In one embodiment, the color is determined using the Royal Horticultural Society (RHS) color chart where colors are arranged in order of the fully saturated colors with the less saturated and less bright colors alongside. The color groups proceed through the observable spectrum and the colors referred to herein are generally in the red-purple (RHSCC 58-74), purple (RHSCC 75-79), purple-violet (RHSCC 81-82), violet (RHSCC 83-88), violet-blue (89-98), blue (RHSCC 99-110) groups contained in Fan 2. Colors are selected from the range including 61A, 64A, 71A, 71C, 72A, 81A, 86A and 87A and colors in between or proximal thereto.

Hence, the present invention is directed to a genetically modified plant including its progeny with purple/violet shades of color comprising a functional non-indigenous F3′,5′H, a functional DFR in petals and genetic material which down regulates expression of a plant's indigenous DFR gene.

In one embodiment, the genetic material comprises sense and anti-sense nucleotide sequences which correspond to the plant's indigenous DFR sequence (ds plantDFR). This induces hairpin RNAi (hpRNAi)-mediated silencing primarily via post-transcriptional gene silencing (PTGS). By “indigenous” is meant that an enzyme or a gene evolved in a plant, i.e. is normally resident in that plant. A “non-indigenous” enzyme or gene means that a gene or other genetic material was introduced into a plant or a parent of the plant by genetic angering or breeding practices.

In an embodiment, the plant is a carnation such as a spray carnation and the indigenous DFR is the carnation DFR. The genetic material is a chimeric construct referred to as ds carnDFR.

In an embodiment, the plant and its progeny, further comprise genetic material encoding a non-indigenous S adenosylmethionine: anthocyanin 3′,5′ methyltransferase (3′5′ AMT) and/or a non-indigenous flavone synthase (FNS).

In a further embodiment the 3′5′ AMT is from Torenia (ThMT) and the FNS is from Torenia (ThFNS).

The modified plants and in particular genetically modified spray carnations comprise genetic sequences encoding at least one F3′5′H enzyme and at least one DFR enzyme and express at least one ds plantDFR molecule. Insofar as the present invention relates to carnations, the ds plantDFR is ds carnDFR and the carnation sprays are conveniently in a Cerise Westpearl genetic background including the progenitor of Cerise Westpearl such as Westpearl. Other carnation cultivars included within the present invention are colored varieties such as Cinderella, Kortina Chanel, Vega, Artisan, Miledy, Barbara, Dark Rendezvous. Other plants contemplated herein include chrysanthemums, roses, gerberas, lisianthus, tulip, lily, geranium, petunia, iris, Torenia, Begonia, Cyclamen, Nierembergia, Catharanthus, Pelargonium, orchid, grape, apple, Euphorbia, Fuchsia and other ornamental or horticultural plants.

One aspect of the present invention is directed to a genetically modified plant exhibiting altered inflorescence in selected tissue, the plant comprising expressed genetic material encoding at least one F3′5′H enzyme and at least one DFR enzyme and expressing genetic material which down regulates a DFR gene. More particularly, the present invention provides a genetically modified plant exhibiting altered inflorescence, the plant or its progeny comprising expressed genetic material encoding at least one non-indigenous F3′5′H enzyme and at least one non-indigenous DFR enzyme and expressing genetic material which down regulates expression of the plant's indigenous DFR gene. In an embodiment, the plant and its progeny, further comprise genetic material encoding a non-indigenous ThMT. In a particular embodiment, the genetic material which down regulates the indigenous DFR gene comprises sense and anti-sense nucleotide sequence corresponding to the indigenous DFR gene or its mRNA (“ds plantDFR”). The term “altered inflorescence” in this context means compared to the inflorescence of a plant (e.g. parent plant or plant of the same species) prior to genetic manipulation. The term “encoding” includes the expression of the genetic material to produce functional F3′5′H and DFR enzymes.

A “ds plantDFR molecule” is genetic material comprising both sense and anti-sense fragments of a plant is indigenous DFR genomic or cDNA sequence or corresponding mRNA. The ds plantDFR is expressed to induce hpRNAi-mediated gene silencing of an indigenous DFR gene. In a particular embodiment, the plant is carnation and the ds plantDFR molecule is ds carnDFR.

In a particular embodiment, the plant is a spray carnation.

Accordingly, another aspect of the present invention is directed to a spray carnation plant exhibiting altered inflorescence in selected tissue, the spray carnation comprising expressed genetic material encoding at least one non-indigenous F3′5′H enzyme and at least one non-indigenous DFR enzyme and expressing at least one ds carnDFR molecule which down regulates expression of the plant's indigenous DFR gene.

Yet another, aspect of the present invention is directed to a genetically modified Cerise Westpearl spray carnation plant or sport thereof exhibiting tissues of a purple to blue color, the carnation comprising expressed genetic sequences encoding at least one non-indigenous F3′5′H enzyme and at least one non-indigenous DFR enzyme and expressing at least one ds carnDFR molecule which down regulates expression of the plant's indigenous DFR gene.

Another aspect of the present invention is directed to a genetically modified chrysanthemum plant exhibiting tissues of a purple to blue color, the chrysanthemum comprising expressed genetic sequences encoding at least one non-indigenous F3′5′H enzyme and at least one non-indigenous DFR enzyme and expressing at least one ds chrysDFR molecule which down regulates expression of the plant's indigenous DFR gene.

Still another aspect of the present invention is directed to a genetically modified rose plant exhibiting tissues of a purple to blue color, the rose comprising expressed genetic sequences encoding at least one non-indigenous F3′5′H enzyme and at least one non-indigenous DFR enzyme and at least one ds roseDFR molecule which down regulates expression of the plant's indigenous DFR gene.

Even yet another aspect of the present invention is directed to a genetically modified gerbera plant exhibiting tissues of a purple to blue color, the gerbera comprising expressed genetic sequences encoding at least one non-indigenous F3′5′H enzyme and at least one non-indigenous DFR enzyme and at least one ds gerbDFR molecule which down regulates expression of the plant's indigenous DFR gene.

Yet another aspect of the present invention is directed to a genetically modified ornamental or horticultural plant exhibiting tissues of a purple to blue color, the ornamental or horticultural plant comprising expressed genetic sequences encoding at least one non-indigenous F3′5′H enzyme and at least one non-indigenous DFR enzyme and incorporation of at least one ds plantDFR molecule which down regulates expression of the plant's indigenous DFR gene.

In an embodiment, the plant and its progeny, further comprise genetic material encoding a non-indigenous ThMT and/or a non-indigenous ThFNS. Reference to “purple to blue” includes mauve.

In a particular embodiment, the present invention provides a genetically modified spray carnation identified herein as Cerise Westpearl (CW)/pCGP3366 and its progeny and sports. In another embodiment, the present invention provides a genetically modified spray carnation identified herein as Cerise Westpearl (CW)/pCGP3601 and its progeny and sports. In yet another embodiment, the present invention provides a genetically modified spray carnation identified herein as Cerise Westpearl (CW)/pCGP3605 and its progeny and sports. Still in another embodiment, the present invention provides a genetically modified spray carnation identified herein as Cerise Westpearl (CW)/pCGP3616 and its progeny and sports. Even in yet another embodiment, the present invention provides a genetically modified spray carnation identified herein as Cerise Westpearl (CW)/pCGP3607 and its progeny and sports.

Progeny, reproductive material, cut flowers, tissue culturable cells and regenerable cells from the genetically plants also form part of the present invention.

The present invention further provides for the use of genetic sequences encoding at least one non-indigenous F3′5′H enzyme and at least one non-indigenous DFR enzyme and genetic material which down regulates a plant's indigenous DFR gene in the manufacture of a carnation or sports thereof exhibiting altered inflorescence including tissue having a purple to violet to blue color.

More particularly, the present invention is directed to the use of genetic sequences encoding at least one non-indigenous F3′5′H enzyme and at least one non-indigenous DFR enzyme and incorporation of at least one ds carnDFR molecule in the manufacture of a genetically modified plant such as a spray carnation including a Cerise Westpearl carnation or sports thereof exhibiting altered inflorescence including tissue having a purple to blue color.

The F3′5′H enzymes may be from any source. Nucleotide sequences encoding F3′5′H enzymes from Viola sp are particularly useful (see Table 1). Similarly, the nucleotide sequence encoding the DFR enzyme may come from any species such as but not limited to Petunia sp (e.g. see Table 1), iris, cyclamen, delphinium, gentian, Cymbidium, nierembergia The sense and anti-sense fragments forming the hairpin loop of the ds carnDFR comes from carnation. The intron in the ds carnDFR comes from petunia DFR-A intron 1 (Beld et al, Plant Mol. Biol. 13:491-502, 1989), however, any intron that is able to be processed in carnation can be used. In another embodiment no intron is used.

Suitable nucleotide sequences for F3′5′H from Viola sp., a DFR from Petunia sp and a DFR from Dianthus sp are set forth in Table 1.

TABLE 1
Summary of sequence identifiers

| SEQ ID | | | TYPE | |
|---|---|---|---|---|
| NO: | NAME | SPECIES | OF SEQ | DESCRIPTION |
| 1 | BPF3′5′H#40.nt | Viola sp | nucleotide | F3′5′H cDNA |
| 2 | BPF3′5′H#40.aa | Viola sp | amino acid | deduced F3′5′H amino acid sequence |
| 3 | Pet gen DFR.nt | Petunia sp | nucleotide | DFR genomic clone |
| 4 | Pet gen DFR.aa | Petunia sp | amino acid | deduced DFR amino acid sequence |
| 5 | DFRint35S F | | nucleotide | primer |
| 6 | DFRint35S R | | nucleotide | primer |
| 7 | ds carnDFR F | | nucleotide | primer |
| 8 | ds carnDFR R | | nucleotide | primer |
| 9 | Carn DFR.nt | Dianthus | nucleotide | DFR cDNA |
| | | caryophyllus | | |
| 10 | Carn DFR.aa | Dianthus | amino acid | deduced DFR amino acid sequence |
| | | caryophyllus | | |
| 11 | ThMT.nt | Torenia sp. | nucleotide | 3′5′ AMT cDNA |
| 12 | ThMT.aa | Torenia sp. | amino acid | deduced 3′5′ AMT amino acid sequence |
| 13 | ThFNS.nt | Torenia sp. | nucleotide | FNS cDNA |
| 14 | ThFNS.aa | Torenia sp. | amino acid | deduced FNS amino acid sequence |
| 15 | carnANS 5′ | Dianthus | nucleotide | Carnation ANS promoter fragment |
| | | caryophyllus | | |
| 16 | carnANS 3′ | Dianthus | nucleotide | Carnation ANS terminator fragment |
| | | caryophyllus | | |
| 17 | RoseCHS 5′ | Rosa hybrida | nucleotide | Rose CHS promoter fragment |

BP, black pansy; nt, nucleotide; aa, amino acid; pet, petunia; carn, carnation; ThMT, S-adenosylmethionine: anthocyanin 3′,5′ methyltransferase from torenia; ANS, anthocyanin synthase; CHS, chalcone synthase; 3′5′ AMT, S-adenosylmethionine: anthocyanin 3′,5′ methyltransferase; FNS, flavone synthase; ThFNS, flavone synthase from torenia.

### BRIEF DESCRIPTION OF THE FIGURES

FIG. 1 is a schematic representation of the biosynthesis pathway for the flavonoid pigments showing production of the anthocyanidin 3-glucosides that occur in most plants that produce anthocyanins. Enzymes involved in the pathway have been indicated as follows: PAL=Phenylalanine ammonia-lyase; C4H=Cinnamate 4-hydroxylase; 4CL=4-coumarate:CoA ligase; CHS=Chalcone synthase; CHI=Chalcone flavanone isomerase; F3H=Flavanone 3-hydroxylase; DFR=Dihydroflavonol-4-reductase; ANS=Anthocyanidin synthase, 3GT=UDP-glucose: flavonoid 3-O-glucosyltransferase; Other abbreviations include: DHK=dihydrokaempferol, DHQ=dihydroquercetin, DHM=dihydromyricetin.

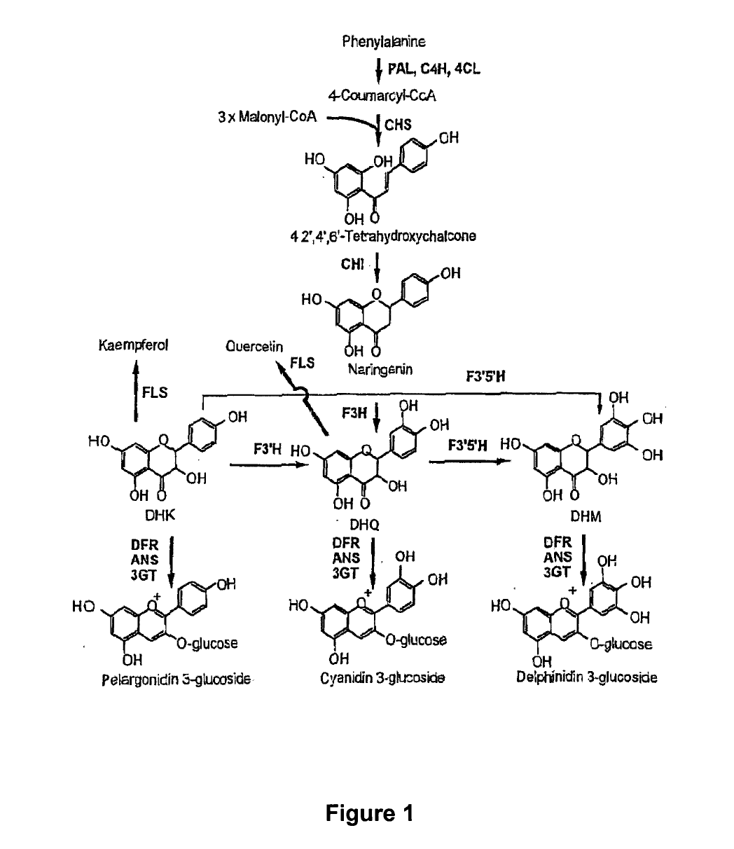

FIG. 2 is a diagrammatic representation of the binary plasmid pCGP3360. chimeric. The construction of pCGP3360 is described in Example 1. Selected restriction endonuclease sites are marked. Abbreviations include LB=Left Border from A. tumefaciens Ti plasmid, RB=Right border region from A. tumefaciens Ti plasmid, TetR=antibiotic, tetracycline resistance gene complex. Refer to Table 2 for a description of gene elements.

FIG. 3 is a diagrammatic representation of the binary plasmid pCGP3366. chimeric. The construction of pCGP3366 is described in Example 1. Selected restriction endonuclease sites are marked. Abbreviations include LB=Left Border from A. tumefaciens Ti plasmid, RB=Right border region from A. tumefaciens Ti plasmid, TetR=antibiotic, tetracycline resistance gene complex. In this figure “ds carnDFR”=the CaMV 35S:ds carnDFR:35S 3′ expression cassette. Refer to Table 2 for a description of gene elements.

FIG. 4 is a diagrammatic representation of the binary plasmid pCGP3601. chimeric. The construction of pCGP3601 is described in Example 1. Abbreviations include LB=Left Border from A. tumefaciens Ti plasmid, RB=Right border region from A. tumefaciens Ti plasmid, TetR=antibiotic, tetracycline resistance gene complex. In this figure “ds carnDFR”=the CaMV 35S:ds carnDFR:35S 3′ expression cassette. Refer to Table 2 for a description of gene elements.

FIG. 5 is a diagrammatic representation of the binary plasmid pCGP3605. chimeric. The construction of pCGP3605 is described in Example 1. Abbreviations include LB=Left Border from A. tumefaciens Ti plasmid, RB=Right border region from A. tumefaciens Ti plasmid, TetR=antibiotic, tetracycline resistance gene complex. In this figure “ds carnDFR”=the CaMV 35S:ds carnDFR:35S 3′ expression cassette and “ThMt”=CaMV 35S:ThMT:35S 3′ expression cassette. Refer to Table 2 for a description of gene elements.

FIG. 6 is a diagrammatic representation of the binary plasmid pCGP3616. chimeric. The construction of pCGP3616 is described in Example 1. Abbreviations include LB=Left Border from A. tumefaciens Ti plasmid, RB=Right border region from A. tumefaciens Ti plasmid, TetR=antibiotic, tetracycline resistance gene complex. In this figure “ds carnDFR”=the CaMV 35S:ds carnDFR:35S 3′ expression cassette. Refer to Table 2 for a description of gene elements.

FIG. 7 is a diagrammatic representation of the binary plasmid pCGP3607. chimeric. The construction of pCGP3607 is described in Example 1. Abbreviations include LB=Left Border from A. tumefaciens Ti plasmid, RB=Right border region from A. tumefaciens Ti plasmid, TetR=antibiotic, tetracycline resistance gene complex. In this figure “ds carnDFR”=the CaMV 35S:ds carnDFR:35S 3′ expression cassette and “ThFNS”=e35S 5′:ThFNS:petD8 3′ expression cassette. Refer to Table 2 for a description of gene elements.

### DETAILED DESCRIPTION

As used in the subject specification, the singular forms “a”, “an” and “the” include plural aspects unless the context clearly dictates otherwise. Thus, for example, reference to “a plant” includes a single plant, as well as two or more plants; reference to “an anther” includes a single anther as well as two or more anthers; reference to “the invention” includes a single aspect or multiple aspects of an invention; and so on.

The present invention contemplates genetically modified plants such as carnation plants and in particular spray carnations exhibiting altered inflorescence. The altered inflorescence may be in any tissue or organelle including flowers, petals, anthers and styles. Particular inflorescence contemplated herein includes a color in the range of red-purple to blue color such as a purple to blue color including mauve. The color determination is conveniently measured against the Royal Horticultural Society (RHS) color chart (RHSCC) and includes colors 77A, 77B, N80B, 81A, 81B, 82A, 82B, 88D and colors in between or proximal to either end of the above range. The term “inflorescence” is not to be narrowly construed and relates to any colored cells, tissues organelles or parts thereof, as well as flowers and petals.

Hence, one aspect of the present invention is directed to a genetically modified plant exhibiting altered inflorescence in selected tissue, the plant comprising expressed genetic material encoding at least one F3′5′H enzyme and at least one DFR enzyme and expressing genetic material which down regulates a plant's indigenous DFR gene. The “plant” includes a parent plant and its progeny which carry on the genetic modification. In particular, the present invention provides a genetically modified plant exhibiting altered inflorescence, the plant or its progeny comprising expressed genetic material encoding at least one non-indigenous flavonoid 3′,5′ hydroxylase (F3′5′H) enzyme and at least one non-indigenous dihydroflavonol 4-reductase (DFR) enzyme and expressing genetic material which down regulates expression of the plant's indigenous DFR gene.

In an embodiment, the plant and its progeny, further comprise genetic material encoding a non-indigenous S-adenosylmethionine: anthocyanin 3′,5′ methyltransferase (ThMT) and/or a flavone synthase (ThFNS). The genetic material which down regulates the plant's indigenous DFR gene comprises, in one embodiment, sense and anti-sense nucleotide sequences corresponding to the plant's indigenous DFR gene or mRNA (ds plantDFR).

The ds plantDFR molecule is a chimeric construct of sense and anti-sense genetic material from the DFR genomic DNA or cDNA corresponding to the indigenous DFR gene or its mRNA in the host plant. The “indigenous” DFR is the DFR normally resident in the host plant prior to genetic manipulation. A non-indigenous enzyme or gene includes a gene or other genetic material which has been introduced into a plant or a parent of the plant by genetic engineering or plant breeding practices.

The ds plantDFR molecule when expressed down-regulates via PTGS the DFR gene in the host plant. The ds plantDFR molecule may be from carnation (ds carnDFR), chrysanthemum (ds chrysDFR), rose (ds roseDFR), gerbera (ds gerbDFR), dianthus (ds dianDFR), petunia (ds petDFR) or from an ornamental or horticultural plant (ds plantDFR). Other ds plantDFR's may come from lisianthus, tulip, lily, geranium, petunia, iris, Torenia, Begonia, Cyclamen, Nierembergia, Catharanthus, Pelargonium, orchid, grape, apple, Euphorbia or Fuchsia.

In a particular embodiment, the plant is a carnation. Accordingly, another aspect of the present invention is directed to a spray carnation exhibiting altered inflorescence in selected tissue, the spray carnation comprising expressed genetic material encoding at least one non-indigenous F3′5′H enzyme and at least one non-indigenous DFR enzyme and expressing of at least one ds carnDFR molecule. The ds carnDFR, when expressed, down regulates expression of the plant's indigenous DFR gene.

In an embodiment, the plant and its progeny, further comprise genetic material encoding a non-indigenous ThMT and/or ThFNS.

Hence, a further aspect of the present invention is directed to a spray carnation exhibiting altered inflorescence in selected tissue, the spray carnation comprising expressed genetic material encoding at least one non-indigenous F3′5′H enzyme, at least one non-indigenous DFR enzyme and at least one non-indigenous ThMT and/or ThFNS and expressing of at least one ds carnDFR molecule.

Whilst the present invention encompasses any spray carnation, a carnation of the Cerise Westpearl line is particularly useful including sports thereof. Useful sports of Cerise Westpearl include Westpearl.

Accordingly, another aspect of the present invention is directed to a genetically modified Cerise Westpearl spray carnation plant line or sports thereof exhibiting tissues of a purple to blue color, the carnation comprising expressed genetic sequences encoding at least one non-indigenous F3′5′H enzyme and at least one non-indigenous DFR enzyme and expressing at least one ds carnDFR molecule which down regulates expression of the plant's indigenous DFR gene.

More particularly, the present invention provides a genetically modified Cerise Westpearl plant (CW)/pCGP3366 (also referred to as CW/3366 or Cerise Westpearl/3366) line exhibiting altered inflorescence, the line comprising an expressed genetic sequence encoding at least one non-indigenous F3′5′H enzyme and at least one non-indigenous DFR enzyme and expressing at least one ds carnDFR molecule which down regulates expression of the plant's indigenous DFR gene.

Even more particularly, the present invention provides a genetically modified Cerise Westpearl plant (CW)/pCGP3601 (also referred to as CW/3601 or Cerise Westpearl/3601) line exhibiting altered inflorescence, the line comprising an expressed genetic sequence encoding at least one non-indigenous F3′5′H enzyme and at least one non-indigenous DFR enzyme and expressing at least one ds carnDFR molecule which down regulates expression of the plant's indigenous DFR gene.

Still more particularly, the present invention provides a genetically modified Cerise Westpearl plant (CW)/pCGP3605 (also referred to as CW/3605 or Cerise Westpearl/3605) line exhibiting altered inflorescence, the line comprising an expressed genetic sequence encoding at least one non-indigenous F3′5′H enzyme and at least one non-indigenous DFR enzyme and expressing at least one ds carnDFR molecule which down regulates expression of the plant's indigenous DFR gene.

Even still more particularly, the present invention provides a genetically modified Cerise Westpearl plant (CW)/pCGP3616 (also referred to as CW/3616 or Cerise Westpearl/3616) line exhibiting altered inflorescence, the line comprising an expressed genetic sequence encoding at least one non-indigenous F3′5′H enzyme and at least one non-indigenous DFR enzyme and expressing at least one ds carnDFR molecule which down regulates expression of the plant's indigenous DFR gene.

Yet more particularly, the present invention provides a genetically modified Cerise Westpearl plant (CW)/pCGP3607 (also referred to as CW/3607 or Cerise Westpearl/3607) line exhibiting altered inflorescence, the line comprising an expressed genetic sequence encoding at least one non-indigenous F3′5′H enzyme and at least one non-indigenous DFR enzyme and expressing at least one ds carnDFR molecule which down regulates expression of the plant's indigenous DFR gene.

In each of the above-mentioned aspects, the plant and its progeny may further comprise genetic material encoding a non-indigenous ThMT and/or ThFNS.

Examples of Cerise Westpearl transgenic lines include #25958 (FLORIGENE Moonberry (Trade mark)) and line #25947 (FLORIGENE Moonpearl (Trade mark)).

Additional genetically modified carnations contemplated herein include the spray carnations Westpearl, Kortina Chanel, Vega, Barbara and Artisan and the standard carnations Cinderella, Dark Rendezvous, Miledy.

Other genetically modified plants contemplated herein include chrysanthemums, roses, gerberas, lisianthus, tulip, lily, geranium, petunia, iris, Torenia, Begonia, Cyclamen, Nierembergia, Catharanthus, Pelargonium, orchid, grape, apple, Euphorbia or Fuchsia and other ornamental or horticultural plants.

Another aspect of the present invention is directed to a genetically modified chrysanthemum plant exhibiting tissues of a purple to blue color, the chrysanthemum comprising expressed genetic sequences encoding at least one non-indigenous F3′5′H enzyme and at least one non-indigenous DFR enzyme and expressing at least one ds chrysDFR molecule which down regulates expression of the plant's indigenous DFR gene.

Still another aspect of the present invention is directed to a genetically modified rose plant exhibiting tissues of a purple to blue color, the rose comprising expressed genetic sequences encoding at least one non-indigenous F3′5′H enzyme and at least one non-indigenous DFR enzyme and expressing at least one ds roseDFR molecule which down regulates expression of the plant's indigenous DFR gene.

Yet another aspect of the present invention is directed to a genetically modified gerbera plant exhibiting tissues of a purple to blue color, the gerbera comprising expressed genetic sequences encoding at least one F3′5′H enzyme and at least one DFR enzyme and expressing at least one ds gerbDFR molecule which down regulates expression of the plant's indigenous DFR gene.

Yet another aspect of the present invention is directed to a genetically modified ornamental or horticultural plant exhibiting tissues of a purple to blue color, the ornamental or horticultural plant comprising expressed genetic sequences encoding at least one non-indigenous F3′5′H enzyme and at least one DFR enzyme and expressing at least one ds plantDFR molecule which down regulates expression of the plant's indigenous DFR gene.

In an embodiment, the plant and its progeny, further comprise genetic material encoding a non-indigenous ThMT and/or ThFNS. The term “purple to blue color” includes mauve.

The ds plantDFR, ds chrysDFR, ds roseDFR, ds gerbDFR, ds petDFR and ds dianDFR comprise sense and anti-sense genomic or cDNA fragments of the gene encoding the host plant's DFR. Expression of this molecule results in down-regulation of the indigenous DFR gene in the host plant. Similar comments apply in relation to ds plantDFR's from other host plants.

The genetic sequence may be a single construct carrying the nucleotide sequences encoding the F3′5′H enzymes and the DFR enzyme or multiple genetic constructs may be employed. In addition, the genetic sequences may be integrated into the genome of a plant cell or it may be maintained as an extra-chromosomal artificial chromosome. Still furthermore, the generation of a spray carnation expressing at least one F3′5′H enzyme and at least one DFR enzyme and expressing at least one ds carnDFR molecule may be generated by recombinant means alone or by a combination of conventional breeding and recombinant DNA manipulation. The genetic sequences are “expressed” in the sense of being operably linked to a promoter and other regulatory sequences resulting in transcription and translation to produce F3′5′H and DFR enzymes.

Hence, another aspect of the present invention contemplates a method for producing a genetically modified plant such as a spray carnation exhibiting altered inflorescence, the method comprising introducing into regenerable cells of a plant such as a spray carnation plant expressible genetic material encoding at least one non-indigenous F3′5′H enzyme and at least one non-indigenous DFR enzyme and incorporation of at least one ds carnDFR molecule which down regulates expression of the plant's indigenous DFR gene and regenerating a plant therefrom or obtaining progeny from the regenerated plant.

In an embodiment, the plant and its progeny, further comprise genetic material encoding a non-indigenous ThMT and/or ThFNS.

Similar methodologies are contemplated herein from chrysanthemums, rose, gerbera and ornamental plants.

The plant may then undergo various generations of growth or cultivation. Hence, reference to a genetically modified spray carnation includes progeny thereof and sister lines thereof as well as sports thereof.

Another aspect of the present invention provides a method for producing a genetically modified plant such as a spray carnation line exhibiting altered inflorescence, the method comprising selecting a plant such as a spray carnation comprising expressible genetic material encoding at least one non-indigenous F3′5′H enzyme and at least one DFR enzyme and incorporation of at least one ds carnDFR molecule which down regulates expression of the plant's indigenous DFR gene and crossing this plant with another plant such as a spray carnation comprising genetic material encoding the other of at least one F3′5′H enzyme and at least one non-indigenous DFR enzyme and incorporation of at least one ds carnDFR molecule and then selecting F1 or subsequent generation plants which express the genetic material.

In an embodiment, the plant and its progeny, further comprise genetic material encoding a non-indigenous ThMT and/or ThFNS.

Nucleotide sequences encoding non-indigenous F3′5′H and DFR enzymes relative to a host plant may be from any source including Viola sp, Petunia sp, Salvia sp, Lisianthus sp, Gentiana sp, Sollya sp, Clitoria sp, Kennedia sp, Campanula sp, Lavandula sp, Verbena sp, Torenia sp, Delphinium sp, Solanum sp, Cineraria sp, Vitis sp, Babiana stricta, Pinus sp, Picea sp, Larix sp, Phaseolus sp, Vaccinium sp, Cyclamen sp, Iris sp, Pelargonium sp, Liparieae, Geranium sp, Pisum sp, Lathyrus sp, Catharanthus sp, Malvia sp, Mucuna sp, Vicia sp, Saintpaulia sp, Lagerstroemia sp, Tibouchina sp, Plumbago sp, Hypocalyptus sp, Rhododendron sp, Linum sp, Macroptilium sp, Hibiscus sp, Hydrangea sp, Cymbidium sp, Millettia sp, Hedysarum sp, Lespedeza sp, Asparagus sp, Antigonon sp, Pisum sp, Freesia sp, Brunella sp or Clarkia sp, etc. For example, in one embodiment, the F3′5′H enzyme comes from Viola sp.

The DFR may come again from the same or different plant species. For example in one embodiment the DFR enzyme comes from petunia. In another embodiment the DFR comes from iris.

The sense and anti-sense fragments forming the hairpin loop of the ds carnDFR comes from carnation (EMBL accession number Z67983, GenBank accession number gi: 1067126) or the functional equivalent from chrysanthemum, rose, gerbera or ornamental plant. Since the aim of the ds carnDFR is to down regulate the indigenous carnation DFR gene via RNAi mediated silencing various fragments of the endogenous carnation DFR sequence may be used (see International Patent Application No. PCT/IB99/00606, Wesley et al, Plant J, 27, 581-590, 2001, Ossowski et al, Plant J, 53, 674-690, 2008). For example, in one embodiment a 300 bp fragment is used in a sense and anti-sense direction. The intron in the ds carnDFR comes from petunia DFR-A intron 1 (Beld et al, Plant Mol. Biol. 13:491-502, 1989), however, any intron that is able to be processed in carnation can be used. In another embodiment, no intron is used. Again, the same comments apply for ds plantDFR molecules generically.

The present invention provides for the use of genetic sequences encoding at least one non-indigenous F3′5′H enzyme and at least one non-indigenous DFR enzyme and incorporation of genetic material which down regulates a plant's indigenous DFR gene in the manufacture of a carnation or sports thereof exhibiting altered inflorescence including tissue having a purple to violet to blue color.

The present invention also contemplates the use of genetic sequences encoding at least one non-indigenous F3′5′H enzyme and at least one non-indigenous DFR enzyme and incorporation of at least one ds carnDFR molecule in the manufacture of a spray carnation plant such as a Cerise Westpearl carnation or sports thereof exhibiting altered inflorescence including tissue having a purple to blue color.

In another embodiment, the present invention contemplates the use of genetic sequences encoding at least one non-indigenous F3′5′H enzyme and at least one non-indigenous DFR enzyme and incorporation of at least one ds DFR (directed at silencing of the indigenous DFR gene) molecule in the manufacture of a genetically modified plant selected from a rose, chrysanthemum, gerbera, tulip, lily, orchid, lisianthus, begonia, torenia, geranium, petunia, nierembergia, pelargonium, iris, impatiens, cyclamen grape, apple, Euphorbia or Fuchsia or other ornamental or horticultural thereof exhibiting altered inflorescence including tissue having a purple to blue color.

In an embodiment, the plant and its progeny, further comprise genetic material encoding a non-indigenous ThMT and/or ThFNS. Plant cells may require to be transformed with two or more genetic constructs each carrying one or more of the various genes. The range “purple to blue color” includes mauve.

Cut flowers, tissue culturable cells, regenerable cells, parts of plants, seeds, reproductive material (including pollen) are all encompassed by the present invention.

As indicated above, nucleotide sequences encoding F3′5′H and DFR enzymes may all come from the same species of plant or from two or more different species. F3′5′H nucleotide sequence from Viola sp and a DFR from a Petunia sp and carnation are particularly useful in the practice of the present invention. The nucleotide sequences encoding the F3′5′H enzymes and the DFR enzymes and the respective amino acid sequences are defined in Table 1.

Nucleic acid molecules encoding F3′5′Hs are also provided in International Patent Application No. PCT/AU92/00334 and Holton et al, 1993 supra. These sequences have been used to modulate 3′,5′ hydroxylation of flavonoids in petunia (see International Patent Application No. PCT/AU92/00334 and Holton et al, 1993 supra), tobacco (see International Patent Application No. PCT/AU92/00334) and carnations (see International Patent Application No. PCT/AU96/00296). Nucleotide sequences of F3′5′H from other species such as Viola, Salvia and Sollya have been cloned (see International Patent Application No. PCT/AU03/01111). Any of these sequences may be used in combination with a promoter and/or terminator. The present invention particularly contemplates F3′5′H encoded by SEQ ID NO:1 and a DFR encoded by SEQ ID NO:3 and a carnation DFR (Z67983, gi: 1067126) (SEQ ID NO:9) or a nucleotide sequence capable of hybridizing to any of SEQ ID NOs:1 or 3 or 9 or a complementary form thereof under low or high stringency conditions or which has at least about 70% identity to SEQ ID NO:1 or 3 or 9 after optimal alignment.

For the purposes of determining the level of stringency to define nucleic acid molecules capable of hybridizing to SEQ ID NO:1, SEQ ID NO:3, SEQ ID NO:9 or their complementary forms, low stringency includes and encompasses from at least about 0% to at least about 15% v/v formamide and from at least about 1M to at least about 2 M salt for hybridization, and at least about 1 M to at least about 2 M salt for washing conditions. Generally, low stringency is from about 25-30° C. to about 42° C. The temperature may be altered and higher temperatures used to replace the inclusion of formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C) % (Marmur and Doty, J. Mol. Biol. 5:109, 1962). However, the Tₘ of a duplex DNA decreases by 1° C. with every increase of 1% in the number of mismatch base pairs (Bonner and Laskey, Eur. J. Biochem. 46:83, 1974). Formamide is optional in these hybridization conditions. Particular levels of washing stringency include as follows: low stringency is 6×SSC buffer, 1.0% w/v SDS at 25-42° C.; a moderate stringency is 2×SSC buffer, 1.0% w/v SDS at a temperature in the range 20° C. to 65° C.; high stringency is 0.2 to 2×SSC buffer, 0.1%-1.0% w/v SDS at a temperature of at least 65° C.

Reference to at least 70% identity includes 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100% identity. The comparison may also be made at the level of similarity of amino acid sequences of SEQ ID NO:s:2, 4 or 10. Hence, nucleic acid molecules are contemplated herein which encode an F3′5′H enzyme or DFR having at least 70% similarity to the amino acid sequence set forth in SEQ ID NOs:2 or 4 10. Again, at least 70% similarity includes 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100% similarity or identity.

The nucleic acid molecule encoding the F3′5′H and DFR enzymes and expression of the ds cam DFR molecule includes one or more promoters and/or terminators. In one embodiment, a promoter is selected which directs expression of a F3′5′H and/or a DFR nucleotide sequence in tissue having a higher pH.

In an embodiment, the promoter sequence is native to the host carnation plant to be transformed or may be derived from an alternative source, where the region is functional in the host plant. Other sources include the Agrobacterium T-DNA genes, such as the promoters for the genes encoding enzymes for biosynthesis of nopaline, octapine, mannopine, or other opines; promoters from plants, such as promoters from genes encoding ubiquitin; tissue specific promoters (see, e.g., U.S. Pat. No. 5,459,252 to Conkling et al; WO 91/13992 to Advanced Technologies); promoters from plant viruses (including host specific viruses), or partially or wholly synthetic promoters. Numerous promoters that are potentially functional in carnation plants (see, for example, Greve, J. Mol. Appl. Genet. 1:499-511, 1983; Salomon et al, EMBO, J. 3:141-146, 1984; Garfinkel et al, Cell 27:143-153, 1983; Barker et al, Plant Mol. Biol. 2:235-350, 1983); including various promoters isolated from plants (such as the Ubi promoter from the maize obi-1 gene, see, e.g., U.S. Pat. No. 4,962,028) and viruses (such as the cauliflower mosaic virus promoter, CaMV 35S). In other embodiments the promoter is AmCHS 5′, RoseCHS 5, carnANS 5′ and/or petDFR 5′ (from Pet gen DFR) with corresponding terminators petD8 3′, nos 3, carn ANS 3′ and petDFR 3′ (from Pet gen DFR), respectively.

The promoter sequences may include cis-acting sequences which regulate transcription, where the regulation involves, for example, chemical or physical repression or induction (e.g., regulation based on metabolites, light, or other physicochemical factors; see, e.g., WO 93/06710 disclosing a nematode responsive promoter) or regulation based on cell differentiation (such as associated with leaves, roots, seed, or the like in plants; see, e.g. U.S. Pat. No. 5,459,252 disclosing a root-specific promoter).

Other cis-acting sequences which may be employed include transcriptional and/or translational enhancers. These enhancer regions are well known to persons skilled in the art, and can include the ATG initiation codon and adjacent sequences.

The nucleic acid molecule(s) encoding at least one F3′5′H enzyme and at least one DFR enzyme and incorporation of at least one ds carnDFR molecule, in combination with suitable promoters and/or a terminators is/are used to modulate the activity of a flavonoid molecule in a spray carnation. Reference herein to modulating the level of a delphinidin-based molecule relates to an elevation or reduction in levels of up to 30% or more particularly of 30-50%, or even more particularly 50-75% or still more particularly 75% or greater above or below the normal endogenous or existing levels of activity.

The term “inflorescence” as used herein refers to the flowering part of a plant or any flowering system of more than one flower which is usually separated from the vegetative parts by an extended internode, and normally comprises individual flowers, bracts and peduncles, and pedicels. As indicated above, reference to a “transgenic plant” may also be read as a “genetically modified plant” and includes a progeny or hybrid line ultimately derived from a first generation transgenic plant.

The present invention also contemplates the use of genetic sequences encoding at least one non-indigenous F3′5′H enzyme and at least one non-indigenous DFR enzyme and incorporation of at least one ds carnDFR molecule which down regulates expression of an indigenous DFR gene in the manufacture of a spray carnation such as a Cerise Westpearl carnation or sports thereof exhibiting altered inflorescence including tissue having a purple to blue color.

The present invention also contemplates the use of genetic sequences encoding at least one non-indigenous F3′5′H enzyme and at least one non-indigenous DFR enzyme and incorporation of at least one ds chrysDFR molecule which down regulates expression of an indigenous DFR gene in the manufacture of a chrysanthemum plant or sports thereof exhibiting altered inflorescence including tissue having a purple to blue color.

The present invention also contemplates the use of genetic sequences encoding at least one non-indigenous F3′5′H enzyme and at least one non-indigenous DFR enzyme and incorporation of at least one ds roseDFR molecule which down regulates expression of an indigenous DFR gene in the manufacture of a rose plant or sports thereof exhibiting altered inflorescence including tissue having a purple to blue color.

The present invention also contemplates the use of genetic sequences encoding at least one non-indigenous F3′5′H enzyme and at least one non-indigenous DFR enzyme and incorporation of at least one ds gerbDFR molecule which down regulates expression of an indigenous DFR gene in the manufacture of a gerbera plant or sports thereof exhibiting altered inflorescence including tissue having a purple to blue color.

The present invention also contemplates the use of genetic sequences encoding at least one non-indigenous F3′5′H enzyme and at least one non-indigenous DFR enzyme and incorporation of at least one ds plantDFR molecule which down regulates expression of an indigenous DFR gene in the manufacture of a plant exhibiting altered inflorescence including tissue having a purple to blue color.

In an embodiment, the plant and its progeny, further comprise genetic material encoding a non-indigenous ThMT and/or ThFNS. The genetic material may comprise a single or multiple constructs. The “purple to blue color” includes mauve.

Similar use embodiments apply to other plants as listed above.

A cultivation business model is also provided, the model comprising generating a genetically modified spray carnation plant as described herein, providing platelets, seeds, regenerable cells, tissue culturable cells or other material to a grower, generating commercial sale numbers of plants, and providing cut flowers to retailers or wholesalers.

The present invention is further described by the following non-limiting Examples. In these Examples, materials and methods as outlined below were employed:

Methods followed were as described in Sambrook et al, Molecular Cloning: A Laboratory Manual, Cold Spring Harbor Laboratories, Cold Spring Harbor, N.Y., USA, 1989 or Sambrook and Russell, Molecular Cloning: A Laboratory Manual 3^{rd} edition, Cold Spring Harbor Laboratories, Cold Spring Harbor, N.Y., USA, 2001 or Plant Molecular Biology Manual (2^{nd} edition), Gelvin and Schilperoot (eds), Kluwer Academic Publisher, The Netherlands, 1994 or Plant Molecular Biology Labfax, Croy (ed), Bios scientific Publishers, Oxford, UK, 1993.

The cloning vectors pBluescript and PCR script were obtained from Stratagene, USA. pCR72.1 was obtained from Invitrogen, USA.

E. coli Transformation

The Escherichia coli strains used were:

### DH5α

supE44,Δ (lacZYA-ArgF)U169, (ø801acZΔM15), hsdR17(rₖ⁻, mₖ⁺),
recA1, endA1, gyrA96, thi-1, relA1, deoR. (Hanahan, J. Mol. Biol. 166:557, 1983)

### XL1-Blue

supE44, hsdR17(rₖ⁻, mₖ⁺), recA1, endA1, gyrA96, thi-1, relA1,
lac⁻,[F′proAB, lacI^{q}, lacZΔM15, Tn10(tet^{R})] (Bullock et al, Biotechniques 5:376, 1987).
BL21-CodonPlus-RIL strain
ompT hsdS(Rb-mB-) dcm+Tet^{r} gal endA Hte [argU ileY leuW Cam^{r}]M15 E. coli is derived from E. coli K12 and has the phenotype Nal^{s}, Str^{s}, Rif^{s}, Thi⁻, Ara⁺, Gal⁺, Mtl⁻, F⁻, RecA⁺, Uvr⁺, Lon⁺.

Transformation of the E. coli strains was performed according to the method of Inoue et al, Gene 96:23-28, 1990.

Agrobacterium tumefaciens Strains and Transformations

The disarmed Agrobacterium tumefaciens strain used was AGL0 (Lazo et al, Bio/technology 9:963-967, 1991).

Plasmid DNA was introduced into the Agrobacterium tumefaciens strain AGL0 by adding 5 μg of plasmid DNA to 100 μL of competent AGL0 cells prepared by inoculating a 50 mL LB culture (Sambrook et al, 1989 supra) and incubation for 16 hours with shaking at 28° C. The cells were then pelleted and resuspended in 0.5 mL of 85% (v/v) 100 mM CaCl₂/15% (v/v) glycerol. The DNA-Agrobacterium mixture was frozen by incubation in liquid N₂ for 2 minutes and then allowed to thaw by incubation at 37° C. for 5 minutes. The DNA/bacterial mix was then placed on ice for a further 10 minutes. The cells were then mixed with 1 mL of LB (Sambrook et al, 1989 supra) media and incubated with shaking for 16 hours at 28° C. Cells of A. tumefaciens carrying the plasmid were selected on LB agar plates containing appropriate antibiotics such as 50 μg/mL tetracycline or 100 μg/mL gentamycin. The confirmation of the plasmid in A. tumefaciens was done by restriction endonuclease mapping of DNA isolated from the antibiotic-resistant transformants.

### DNA Ligations

DNA ligations were carried out using the Amersham Ligation Kit or Promega Ligation Kit according to procedures recommended by the manufacturer.

### Isolation and Purification of DNA Fragments

Fragments were generally isolated on a 1% (w/v) agarose gel and purified using the QIAEX II Gel Extraction kit (Qiagen) or Bresaclean Kit (Bresatec, Australia) following procedures recommended by the manufacturer.

Repair of Overhanging Ends after Restriction Endonuclease Digestion

Overhanging 5′ ends were repaired using DNA polymerase I Klenow fragment according to standard protocols (Sambrook et al, 1989 supra). Overhanging 3′ ends were repaired using Bacteriophage T4 DNA polymerase according to standard protocols (Sambrook et al, 1989 supra).

Removal of Phosphoryl Groups from Nucleic Acids

Shrimp alkaline phosphatase (SAP) [USB] was typically used to remove phosphoryl groups from cloning vectors to prevent re-circularization according to the manufacturer's recommendations.

### Polymerase Chain Reaction (PCR)

Unless otherwise specified, PCR conditions using plasmid DNA as template included using 2 ng of plasmid DNA, 100 ng of each primer, 2 μL, 10 mM dNTP mix, 5 μL 10×Taq DNA polymerase buffer, 0.5 μL Taq DNA Polymerase in a total volume of 50 μL. Cycling conditions comprised an initial denaturation step of 5 minutes at 94° C., followed by 35 cycles of 94° C. for 20 sec, 50° C. for 30 sec and 72° C. for 1 minute with a final treatment at 72° C. for 10 minutes before storage at 4° C.

PCRs were performed in a Perkin Elmer GeneAmp PCR System 9600.

### ³²P-Labeling of DNA Probes

DNA fragments (50 to 100 ng) were radioactively labeled with 50 μCi of [α-³²P]-dCTP using a Gigaprime kit (Geneworks). Unincorporated [α-³²P]-dCTP was removed by chromatography on Sephadex G-50 (Fine) columns or Microbiospin P-30 Tris chromatography columns (BioRad).

### Plasmid Isolation

Single colonies were analyzed for inserts by inoculating LB broth (Sambrook et al, 1989 supra) with appropriate antibiotic selection (e.g. 100 μg/mL ampicillin or 10 to 50 μg/mL tetracycline etc.) and incubating the liquid culture at 37° C. (for E. coli) or 29° C. (for A. tumefaciens) for ˜16 hours with shaking. Plasmid DNA was purified using the alkali-lysis procedure (Sambrook et al, 1989 supra) or using The WizardPlus SV minipreps DNA purification system (Promega) or Qiagen Plasmid Mini Kit (Qiagen). Once the presence of an insert had been determined, larger amounts of plasmid DNA were prepared from 50 mL overnight cultures using the alkali-lysis procedure (Sambrook et al, 1989 supra) or QIAfilter Plasmid Midi kit (Qiagen) and following conditions recommended by the manufacturer.

### DNA Sequence Analysis

DNA sequencing was performed using the PRISM (trademark) Ready Reaction Dye Primer Cycle Sequencing Kits from Applied Biosystems. The protocols supplied by the manufacturer were followed. The cycle sequencing reactions were performed using a Perkin Elmer PCR machine (GeneAmp PCR System 9600). Sequencing runs were generally performed by the Australian Genome Research Facility at the University of Queensland, St Lucia, Brisbane, Australia and at The Walter and Eliza Hall Institute of Medical Research, Melbourne, Australia.

Sequences were analyzed using a MacVector (Trade mark) application (version 9.5.2 and earlier) [MacVector Inc, Cary, N.C., USA].

Homology searches against Genbank, SWISS-PROT and EMBL databases were performed using the FASTA and TFASTA programs (Pearson and Lipman, Proc. Natl. Acad. Sci. USA 85(8):2444-2448, 1988) or BLAST programs (Altschul et al, J. Mol. Biol. 215(3):403-410, 1990). Percentage sequence similarities were obtained using LALIGN program (Huang and Miller, Adv. Appl. Math. 12:373-381, 1991) or ClustalW program (Thompson et al, Nucleic Acids Research 22:4673-4680, 1994) within the MacVector (Trade mark) application (MacVector Inc, USA) using default settings.

Multiple sequence alignments were produced using ClustalW (Thompson et al, 1994 supra) using default settings.

### Plant Transformations

Plant transformations were as described in International Patent Application No. PCT/US92/02612 incorporated herein by reference or International Patent Application No. PCT/AU96/00296 or Lu et al, Bio/Technology 9:864-868, 1991. Other methods may also be employed.

Cuttings of Dianthus caryophyllus cv. Cerise Westpearl were obtained from Propagation Australia, Queensland, Australia.

### Transgenic Analysis

### Color Coding

The Royal Horticultural Society's Color Charts, Third and/or Fifth edition (London, UK), 1995 and/or 2007 were used to provide a description of observed color. They provide an alternative means by which to describe the color phenotypes observed. The designated numbers, however, should be taken only as a guide to the perceived colors and should not be regarded as limiting the possible colors which may be obtained.

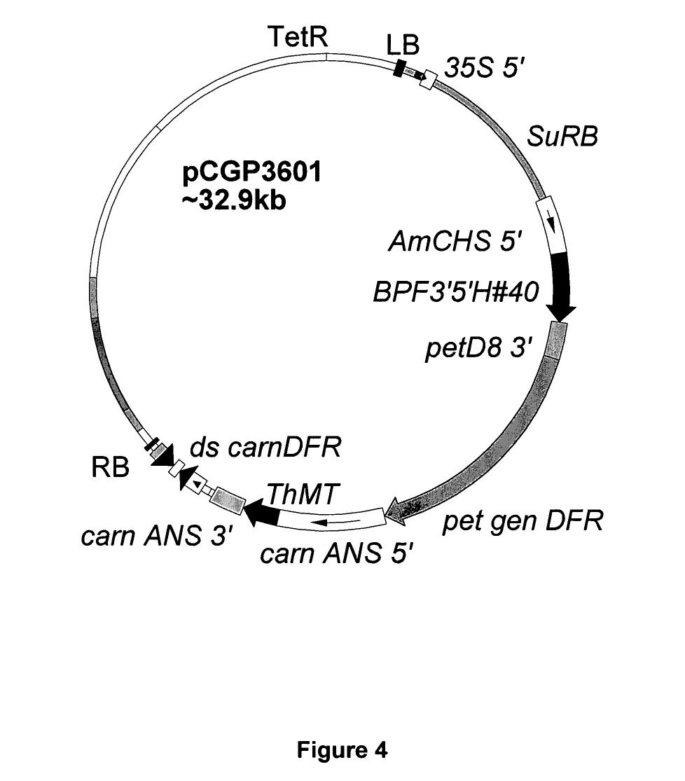

Carnation petals consist of 3 zones, the claw, corona and limb (Glimn-Lacy and Kaufman, Botany Illustrated, Introduction to Plants, Major Groups, Flowering Plant Families, 2^{nd} ed, Springer, USA, 2006). In general only the petal limb is colored with the claw being a green color and the corona a white shade (see FIG. 4). Reference to carnation petal/flower/inflorescence color generally relates to the color of the carnation petal limb.

### Chromatographic Analysis

Thin Layer Chromatography (TLC) and High Performance Liquid Chromatography (HPLC) analysis was performed generally as described in Brugliera et al, Plant J. 5:81-92, 1994.

In general TLC and HPLC analysis was performed on extracts isolated from the petal limbs.

### Extraction of Anthocyanidins

Prior to HPLC analysis, the anthocyanin and flavonol molecules present in petal limb extracts were acid hydrolyzed to remove glycosyl moieties from the anthocyanidin or flavonol core. Anthocyanidin and flavonol standards were used to help identify the compounds present in the floral extracts.

Petal extracts were prepared essentially as described in Fukui et al, 2003 supra. Petal were added to 6 N HCl (0.2 mL) and boiled at 100° C. for 20 min. The hydrolyzed anthocyanidins were extracted with 0.2 mL of 1-pentanol. HPLC analysis of the anthocyanidins was performed using an ODS-A312 (15 cm×6 mm, YMC Co., Ltd, Kyoto, Japan) column, a flow rate of solvent of 1 mL min⁻¹, and detection at an absorbance of 600-400 nm on a SPD-M20A photodiode array detector (Shimadzu Co., Ltd). The solvent system used was as follows: acetic acid:methanol:water=15:20:65. Under these HPLC conditions, the retention time and λₘₐₓ of delphinidin were 4.0 min and 534 nm, respectively, and these values were compared with those of authentic delphinidin chloride (Funakoshi Co., Ltd, Tokyo, Japan).

The anthocyanidin peaks were identified by reference to known standards, viz delphinidin, petunidin, malvidin, cyanidin and peonidin

### Stages of Flower Development

Carnation flowers were harvested at developmental stages defined as follows:

Stage 1: Closed bud, petals not visible.
Stage 2: Flower buds opening: tips of petals visible.
Stage 3: Tips of nearly all petals exposed. “Paint-brush stage”.
Stage 4: Outer petals at 45° angle to stem.
Stage 5: Flower fully open.

For TLC or HPLC analysis, petal limbs were collected from stage 4 flowers at the stage of maximum pigment accumulation.

For Northern blot analysis, petals were collected from stage 3 flowers at the stage of maximal expression of flavonoid pathway genes.

### Example 1

### Preparation of Chimeric F3′5′H Gene Constructs

A summary of promoter, terminator and coding fragments used in the preparation of constructs and the respective abbreviations is listed in Table 2.

TABLE 2
Abbreviations used in construct preparations

| ABBREVIATION | DESCRIPTION |
|---|---|
| CaMV 35S | ~0.4 kb fragment containing the promoter region from |
| | the Cauliflower Mosaic Virus 35S (CaMV 35S) gene - |
| | (Franck et al, I 21: 285-294, 1980, Guilley et al, |
| | Cell, 30: 763-773. 1982) |
| 35S 5′ | promoter fragment from CaMV 35S gene (Franck et |
| | al, 1980 supra) with an ~60 bp 5′ untranslated leader |
| | sequence (CabL) from the petunia chlorophyll a/b |
| | binding protein gene (Cab 22 gene) [Harpster et al, |
| | MGG, 212: 182-190, 1988] |
| AmCHS 5′ | Promoter fragment from the Antirrhinum majus |
| | chalcone synthase (CHS) gene which includes 1.2 kb |
| | sequence 5′ of the translation initiation site (Sommer |
| | and Saedler, Mol Gen. Gent., 202: 429-434, 1986) |
| BPF3′5′H#40 | Viola (Black Pansy) F3′5′H cDNA clone #40 |
| | (International Patent Application No. PCT/AU03/ |
| | 01111 incorporated herein by reference) |
| | (SEQ ID NO: 1) |
| 35S 3′ | ~0.2 kb terminator fragment from CaMV 35S gene |
| | (Franck et al, 1980 supra) |
| Pet gen DFR | ~5.3 kb Petunia DFR-A genomic clone with it's own |
| | promoter and terminator (SEQ ID NO: 3) |
| petD8 3′ | ~0.7 kb terminator region from a phospholipid transfer |
| | protein gene (D8) of Petunia hybrida cv. OGB includes |
| | a 150 bp untranslated region of the transcribed region |
| | of PLTP gene (Holton, Isolation and characterization of |
| | petal-specific genes from Petunia hybrida. PhD Thesis, |
| | University of Melbourne, 1992) |
| SuRB | Herbicide (Chlorsulfuron)-resistance gene (encodes |
| | Acetolactate Synthase) with its own terminator (tSuRB) |
| | from Nicotiana tabacum (Lee et al, EMBO J. 7: 1241- |
| | 1248, 1988) |
| ds carnDFR | “double stranded (ds) carnation DFR” fragment |
| | harboring a ~0.3 kb sense partial carnation DFR cDNA |
| | fragment: 180 bp petunia DFR-A intron 1 fragment |
| | (Beld et al, 1989 supra): ~0.3 kb anti-sense partial |
| | carnation DFR fragment with the aim of formation of |
| | double stranded (hairpin loop) RNA molecule to induce |
| | RNAi-mediated silencing of the endogenous carnation |
| | DFR. The sequence of a complete carnation DFR clone |
| | (Z67983, gi: 1067126) is shown in SEQ ID NO: 9. |
| ThMT | ~1.0 kb cDNA clone corresponding to |
| | S-adenosylmethionine: anthocyanin 3′ 5′ |
| | methyltransferase from torenia (International Patent |
| | Application No. PCT/AU03/00079 incorporated |
| | herein by reference) (SEQ ID NO: 11) |
| ThFNS | ~1.7 kb cDNA clone corresponding to flavone synthase |
| | from torenia (Akashi et al., Plant Cell Physiol. 40 (11): |
| | 1182-1186, 1999, International Patent Application No. |
| | PCT/JP00/00490 incorporated herein by reference) |
| | (SEQ ID NO: 13) |
| carnANS 5′ | Promoter sequence of anthocyanidin synthase (ANS) |
| | gene from Dianthus caryophyllus (See International |
| | Patent Application No. PCT/GB99/02676 incorporated |
| | herein by reference) (SEQ ID NO: 15) |
| carnANS 3′ | Terminator sequence of anthocyanidin synthase gene |
| | (ANS) from Dianthus caryophyllus (See International |
| | Patent Application No. PCT/GB99/02676 incorporated |
| | herein by reference) (SEQ ID NO: 16) |
| RoseCHS 5′ | ~2.8 kb fragment containing the promoter region from |
| | a CHS gene of Rosa hybrida (see International Patent |
| | Application No. PCT/AU03/01111 incorporated herein |
| | by reference) (SEQ ID NO: 17) |
| e35S 5′ | ~0.7 kb fragment incorporating an enhanced CaMV |
| | 35S promoter (Mitsuhashi et al. Plant Cell Physiol. |
| | 37: 49-59, 1996) |

Cerise Westpearl is a cerise colored carnation (RHSCC 57D) It typically accumulates pelargonidin-based pigments (˜99% of total anthocyanin content of 1.0 mg/g petal fresh weight) and therefore lacks F3′H activity and so is presumed mutant in the F3′H gene. HPLC analysis results on 2 flowers revealed 1.08 mg/g anthocyanin (99% pelargonidin), 2.9 to 4.6 mg/g flavonols and 0.3 to 0.6 mg/g dihydroflavonols accumulating in the petals of Cerise Westpearl. Cerise Westpearl is a sport of the pink colored flower Westpearl.

In order to produce novel purple/blue flowers in the spray carnation background of Cerise Westpearl, two binary vector constructs were prepared utilizing the pansy F3′5′H cDNA clone and petunia genomic DFR gene with or without a ds carnDFR expression cassette.

Table 3 provides a summary of chimeric F3′5′H and DFR gene expression cassettes contained in binary vector constructs used in the transformation of Cerise Westpearl (see Table 2 for an explanation of abbreviations).

TABLE 3
Summary of Chimeric Constructs

| Construct | ds plantDFR | DFR | F3′5′H | Other |
|---|---|---|---|---|
| pCGP3360 | none | Pet gen DFR | AmCHS 5′: | |
| | | | BPF3′5′H#40: | |
| | | | petD8 3′ | |
| pCGP3366 | CaMV35S: | Pet gen DFR | AmCHS 5′: | |
| | ds carn DFR: | | BPF3′5′H#40: | |
| | 35S 3′ | | petD8 3′ | |
| pCGP3601 | CaMV35S: | Pet gen DFR | AmCHS 5′: | carnANS 5′: |
| | ds carn DFR: | | BPF3′5′H#40: | ThMT: |
| | 35S 3′ | | petD8 3′ | carnANS 3′ |
| pCGP3605 | CaMV35S : | Pet gen DFR | AmCHS5′: | CaMV 35S: |
| | ds carn DFR: | | BPF3′5′H#40: | ThMT: |
| | 35S 3′ | | petD8 3′ | 35S 3′ |
| pCGP3616 | CaMV35S: | Pet gen DFR | AmCHS 5′: | RoseCHS 5′: |
| | ds carn DFR: | | BPF3′5′H#40: | ThFNS: |
| | 35S 3′ | | petD8 3′ | nos 3′ |
| pCGP3607 | CaMV35S: | Pet gen DFR | AmCHS 5′: | e35S 5′: |
| | ds carn DFR: | | BPF3′5′H#40: | ThFNS: |
| | 35S 3′ | | petD8 3′ | petD8 3′ |

NB All have ALS selectable marker gene (35S 5′:SuRB)
Refer to Table 2 for a description of abbreviations and genetic elements.

The constructs pCGP3601, 3605, 3607, 3616 are all based upon pCGP3366 and have an extra expression cassette that is either a floral specific or constitutive expression of anthocyanin 3′S′ methyltransferase cDNA clone from torenia (targeting methylating of the delphinidin) [pCGP3601 and 3605] or floral specific or constitutive expression of a flavone synthase cDNA clone from torenia (targeting producing of the co-pigments, flavones) [pCGP3616 and 3607].

### Preparation of the Constructs

The Transformation Vector pCGP3360 (AmCHS 5′:BPF3′5′H#40:petD8 3′; Pet gen DFR; 35S 5′:SuRB)

The transformation vector pCGP3360 contains the AmCHS 5′:BPF3′5′H#40: petD8 3′ expression cassette and the petunia genomic DFR-A gene along with the 35S 5′: SuRB selectable marker gene.

Construction of the Intermediate Plasmid, pCGP3356 (AmCHS 5′:BPF3′5′H#40:pet D8 3)

The plasmid pCGP3356 contains a chimeric gene consisting of AmCHS 5′: BPF3′5′H#40:petD8 3′ in a pBluescript backbone.

A ˜1.6 kb fragment harboring the BPF3′5′H#40 cDNA clone was released from the plasmid pCGP1961 (see International Patent Application No. PCT/AU03/01111) upon digestion with the restriction endonucleases EcoRI and KpnI. The overhanging ends were repaired and the fragment was purified. The plasmid pCGP725 containing AmCHS 5′: petHf1:petD8 3′ in pBluescript (described in International Patent Application No. PCT/AU03/01111) was digested with the restriction endonucleases XbaI and BamHI to release the backbone vector harboring the AmCHS 5′ and petD8 3′ regions. The overhanging ends were repaired and the ˜4.9 kb fragment was isolated, purified and ligated with the blunt ended BPF3′5′H#40 fragment from pCGP1961 (described above). Correct insertion of the BPF3′5′H#40 cDNA clone in a sense orientation between the Am CHS 5′ promoter and the pet D8 3′ terminator was established by restriction endonuclease analysis of plasmid DNA isolated from ampicillin-resistant transformants. The resulting plasmid was designated as pCGP3356.

Construction of the Intermediate Plasmid, pCGP3357 (AmCHS 5′:BPF3′5′H#40:pet D8 3′ in pCGP1988)

The plasmid pCGP3357 contains a chimeric gene consisting of AmCHS 5′:

BPF3′5′H#40:petD8 3′ along with the 35S 5′:SuRB selectable marker gene in the pCGP1988 vector (see International Patent Application No. PCT/AU03/01111).

The plasmid pCGP3356 (described above) was digested with the restriction endonuclease PstI to release a 3.5 kb fragment bearing the AmCHS 5′:BPF3′5′H#40: petD8 3′ expression cassette. The resulting 5′-overhang was repaired using DNA Polymerase I (Klenow fragment) according to standard protocols (Sambrook et al, 1989 supra). The fragment was purified and ligated with SmaI ends of the plasmid pCGP1988 (see International Patent Application No. PCT/AU03/01111). Correct insertion of AmCHS 5′:BPF3′5′H#40:petD8 3′ gene in a tandem orientation with respect to the 35S 5′:SuRB selectable marker gene cassette was established by restriction endonuclease analysis of plasmid DNA isolated from tetracycline-resistant transformants. The resulting plasmid was designated as pCGP3357.

Construction of the Intermediate Plasmid, pCGP1472 (Petunia DFR-A Genomic Clone)

A genomic library was made from Petunia hybrida cv. Old Glory Blue DNA in the vector λ2001 (Holton, 1992 supra). Approximately 200,000 pfu were plated out on NZY plates, lifts were taken onto NEN filters and the filters were hybridized with 400,000 cpm/mL of ³²P-labeled petunia DFR-A cDNA fragment (described in Brugliera et al, 1994, supra). Hybridizing clones were purified, DNA was isolated from each and mapped by restriction endonuclease digestion. A 13 kb Sad fragment of one of these clones was isolated and ligated with Sad ends of pBluescriptII to create the plasmid pCGP1472. Finer mapping indicated that an ˜5.3 kb BglII fragment contained the entire petunia DFR-A gene (Beld et al, 1989 supra).

Construction of the Transformation Vector, pCGP3360

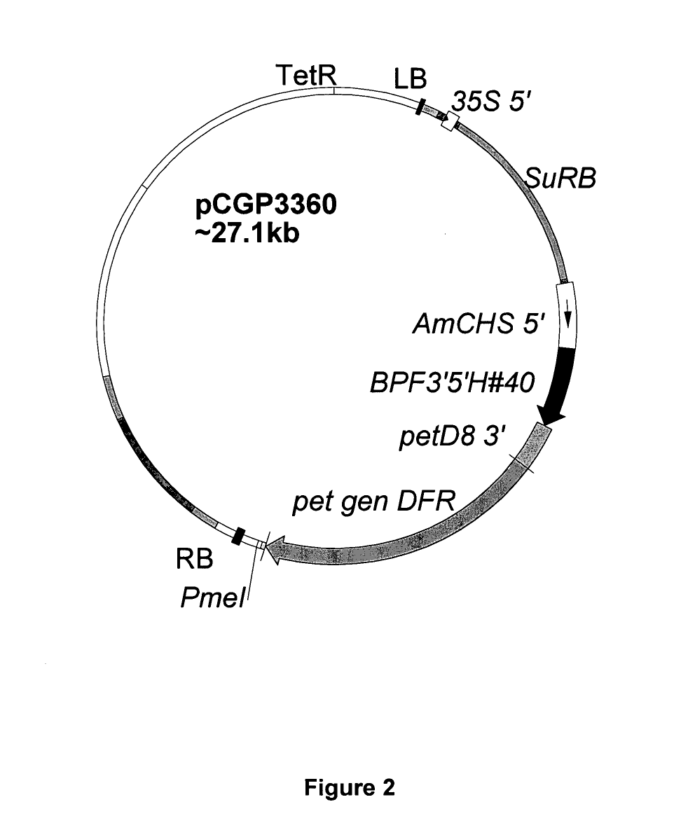

The 5.3 kb fragment harboring the pet gen DFR gene was released from the plasmid pCGP1472 upon digestion with the restriction endonuclease BglII. The overhanging ends were repaired and the fragment was purified and ligated with the repaired AscI ends of the plasmid pCGP3357 (described above). Correct insertion of pet gen DFR gene in a tandem orientation with respect to the AmCHS BPF3′5′H#40:petD8 3′ and 35S 5′:SuRB genes was established by restriction endonuclease analysis of plasmid DNA isolated from tetracycline-resistant transformants. The resulting plasmid was designated as pCGP3360 (FIG. 2).

The Transformation Vector pCGP3366 (CaMV35S:ds carn DFR:35S 3′; Pet gen DFR; AmCHS 5′:BPF3′5′H#40:petD8 3; 35S 5′:SuRB)

The transformation vector pCGP3366 contains the AmCHS 5′:BPF3′5′H#40: petD8 3′ expression cassette and the petunia genomic DFR-A (pet gen DFR) genes along with a CaMV35S:ds carn DFR:35S 3′ expression cassette and the 35S 5′:SuRB selectable marker gene.

Construction of the Intermediate Plasmid pCGP3359

A fragment bearing 180 bp of the petunia DFR-A intron 1 was amplified by PCR using the plasmid pCGP1472 (described above) as template and the following primers:

```
DFRint35S F
(SEQ ID NO: 5)
GCAT CTCGAG GGATCC TCG TGA TCC TGG TAT GTT TTG
            XhoI BamHI
DFRint35S R
(SEQ ID NO: 6)
GCAT TCTAGA AGATCT CTT CTT GTT CTC TAC AAA ATC
            BglII BamHI
```

The forward primer (DFRint35S F) was designed to incorporate the restriction endonuclease recognition sites XhoI and BamHI at the 5′-end. The reverse primer (DFRint35S R) was designed to incorporate Xba I and BglII restriction endonuclease recognition sites at the 3′-end of the 180 bp product that was amplified. The resulting 180 by PCR product was then digested with the restriction endonucleases XhoI and XbaI and ligated with XhoI/XbaI ends of the plasmid pRTppoptcAFP (a source of the CaMV35S promoter and terminator fragments) (Wnendt et al., Curr Genet. 25: 510-523, 1994). Correct insertion of the petunia DFR-A intron 1 fragment between the CaMV35S and 35S 3′ fragments of pRTppoptcAFP was confirmed by restriction endonuclease analysis of plasmid DNA isolated from ampicillin-resistant transformants. The resulting plasmid was designated pCGP3359.

Isolation of Full-Length Carnation DFR cDNA Clone

Isolation of a partial carnation DFR cDNA clone has been described in International Patent Application No. PCT/AU96/00296.

Around 120,000 pfus of a carnation Kortina Chanel petal cDNA library (construction of which is described in International Patent Application No. PCT/AU97/000124) were screened using the ³²P-labeled fragments of an EcoRI/XhoI partial carnation DFR fragment (see International PCT/AU96/00296) as a probe under high stringency hybridization washing conditions. Around 20 strongly hybridizing plaques were selected and further purified. Of these one (KCDFR#17) contained a 1.3 kb insert and represented a full-length carnation DFR cDNA clone with 51 bp of 5′ untranslated sequence. The plasmid was designated as pCGP1547.

Construction of the Intermediate Plasmid pCGP3363 (CaMV35S: Sense Partial Carnation DFR: Petunia DFR Intron 1:35S 3)

A fragment bearing ˜300 bp of the carnation DFR cDNA clone was amplified by PCR using the plasmid pCGP1547 (described above) as template and the following primers:

```
ds carnDFR F
(SEQ ID NO: 7)
GCAT TCTAGA CTCGAG CGA GAA TGA GAT GAT AAA ACC
            Xbal Xhol
ds carnDFR R
(SEQ ID NO: 8)
GCAT AGATCT GGATCC GAG ATT GTT TTC TGC TGC G
            BglII BamHI
```

The forward primer (ds carnDFR F) was designed to incorporate the restriction endonuclease recognition sites XbaI and XhoI at the 5′-end. The reverse primer (ds carnDFR R) was designed to incorporate BglII and BamHI restriction endonuclease recognition sites at the 3′-end of the ˜300 bp product that was amplified. The resulting ˜300 bp PCR product was then digested with the restriction endonucleases XhoI and BamHI and ligated with XhoI/BamHI ends of the plasmid pCGP3359 (described above). Correct insertion of the partial carnation DFR fragment in a sense direction between the CaMV35S and petunia DFR intron 1 fragment of the plasmid pCGP3359 was confirmed by restriction endonuclease analysis of plasmid DNA isolated from ampicillin-resistant transformants. The resulting plasmid was designated pCGP3363.

Construction of the Intermediate Plasmid pCGP3364 (CaMV35S:ds Carn DFR:35S 39

The amplified partial carnation DFR fragment described above was digested with the restriction endonucleases BglII and XbaI and ligated with BglII/XbaI ends of the plasmid pCGP3363 (described above). Correct insertion of the partial carnation DFR fragment in an anti-sense direction between the petunia DFR intron 1 and 35S 3′ fragments of the plasmid pCGP3363 was confirmed by restriction endonuclease analysis of plasmid DNA isolated from ampicillin-resistant transformants. The resulting plasmid was designated pCGP3364.

Construction of the Transformation Vector, pCGP3366

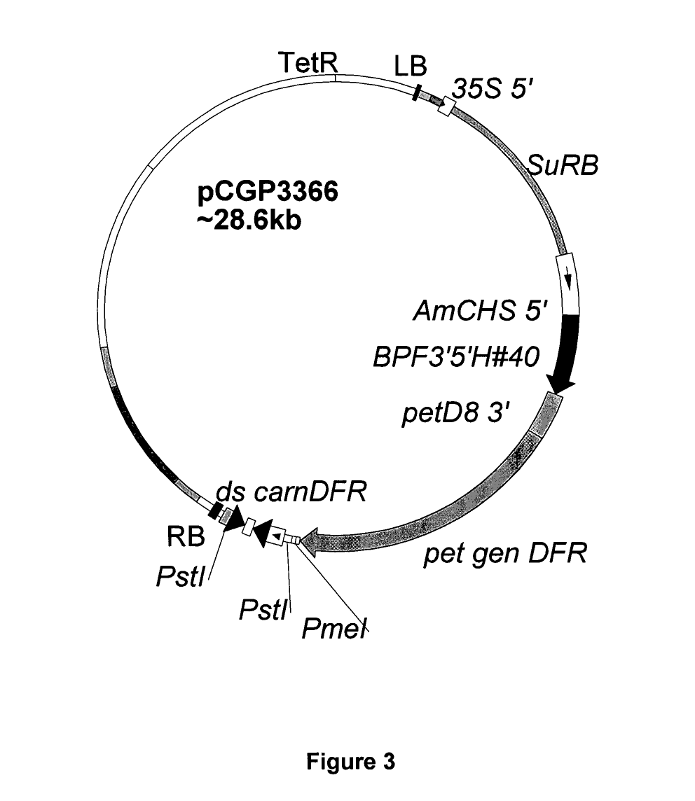

A ˜1.4 kb fragment bearing the CaMV35S:ds carn DFR:35S 3′ expression cassette was released from the plasmid pCGP3364 (described above) upon digestion with the restriction endonuclease PstI. The fragment was purified and ligated with the PstI ends of the plasmid pCGP3360 (described above) (FIG. 2). Correct insertion of CaMV35S:ds carn DFR:35S 3′ expression cassette in a tandem orientation with respect to the AmCHS 5′:BPF3′5′H#40:petD8 3; pet gen DFR and 35S 5′:SuRB genes was established by restriction endonuclease analysis of plasmid DNA isolated from tetracycline-resistant transformants. The resulting plasmid was designated as pCGP3366 (FIG. 3).

The T-DNAs of the transformation vectors pCGP3360 and pCGP3366 were introduced into the spray carnation line, Cerise Westpearl via Agrobacterium-mediated transformation. Transgenic cells were selected based on their ability to grow and produce roots on media containing the herbicide, chlorsulfuron. Transgenic plantlets with roots were removed form media and transferred to soil and grown to flowering in temperature controlled greenhouses in Bundoora, Victoria, Australia.

The color of the petal limbs of the transgenic plants were recorded by eye using RHSCC and HPLC analysis was used to determine the anthocyanidins in the hydrolyzed petal limb extracts. The results are summarized in Table 4.

TABLE 4
Results of transgenic analysis of petals from Cerise Westpearl carnations transformed
with T-DNAs containing F3′5′H and DFR gene expression cassettes.

| | | | | # | % del | | Del |
|---|---|---|---|---|---|---|---|
| transgenes | pCGP | #tg | % CC | HPLC | (Range) | Av del | mg/g FW |
| AmCHS 5′: BP F3′5′H #40: petD8 | 3360 | 38 | 57% | 13 | 52 to 76% | 65% | 0.42 to 1.98 |
| 3′; | | | | | | | |
| Pet gen DFR | | | | | | | |
| AmCHS 5′: BP F3′5′H #40: petD8 | 3366 | 47 | 94% | 34 | 51 to 93% | 84% | 0.28 to 2.68 |
| 3′; | | | | | | | |
| Pet gen DFR; | | | | | | | |
| CaMV 35S: ds carnDFR: 35S 3′ | | | | | | | |

Transgenes = chimeric F3′5′H and DFR nucleotide sequences contained on the T-DNA
pCGP = plasmid pCGP identification number of the transformation vector used in the transformation experiment (refer to Table 3 for details)
#tg = total number of transgenic carnation lines produced
% CC = the percentage of the total number of events produced that had a shift in petal color towards the purple range
# HPLC = number of individual events of which the anthocyanidins of hydrolyzed petal limb extracts were analyzed by HPLC. Petals for analysis were selected based on a visible shift in color of the petal from pink into the purple range.
% del (range) = the range in % of delphinidin detected in the hydrolyzed extracts of the petals for the population of transgenic events
Av del = the average % of delphinidin detected in the hydrolyzed extracts of the petals for the population of transgenic events
Del mg/g FW = the range in the amount of delphinidin (in mg/g of fresh weight) detected in the hydrolyzed extracts of the petals for the population of transgenic events

The results suggest that of the two constructs tested (pCGP3360 and pCGP3366), pCGP3366 resulted in a higher percentage of events that produced flowers with a shift in color to the purple range. Furthermore the average delphinidin detected in the hydrolyzed extracts of the petals was higher in pCGP3366 events compared to pCGP3360 events. This was presumably due to the down regulation of the endogenous carnation DFR by the ds carnDFR cassette via RNAi-mediated silencing leading to decreased competition between the endogenous DFR and the introduced F3′5′H for the DHK substrate. The introduced petunia DFR (which is not able to utilise DHK) subsequently allowed conversion DHM (product of F3′5′H reaction on DHK) to leucodelphinidin and activity by the endogenous anthocyanin pathway enzymes resulted in delphinidin derived pigments accumulating in the petal tissue. To identify spray carnation lines producing petals of a novel color, the colors of petal limbs were compared to mauve/purple carnation lines already in the market place. These included the midi carnation lines FLORIGENE Moonshadow (Trade mark) [82A, 82B] and FLORIGENE Moondust (76A) and the standard carnation lines FLORIGENE Moonvista (Trade mark) [81A+], FLORIGENE Moonshade (Trade mark) [81A, 82A], FLORIGENE Moonlite (Trade mark) [77D/82D, 77C, N80B] and FLORIGENE Moonaqua (Trade mark) [84A/B]. Twenty two CW/3366 lines were initially selected as being novel spray carnation lines whilst only one CW/3360 line was selected as being novel spray carnation line. Further trailing with respect to petal color consistency and petal number reduced the list to 11 CW/3366 lines and no CW/3360 lines as being novel spray carnation lines with potential for new product lines (Table 5).

TABLE 5
RHS color code of the petal limb and delphinidin levels detected in
selected Cerise Westpearl/3366 lines

| ACCESSION | RHSCC | Delphinidin levels | |
|---|---|---|---|
| NUMBER | NUMBER | %, (mg/g FW) | |
| 25930 | 77A | 92% | (2.2 mg/g) |
| 25931 | 77A+ | 93% | (1.7 mg/g) |
| 25932 | 77A+ | 93% | (2.3 mg/g) |
| 25946 | 81B/82B | 84% | (0.3 mg/g) |
| 25947 | 77D, 78D | nd | |
| 25958 | 81B, 82A, N80B | 81% | (0.5 mg/g) |
| 25961 | 77B, 88D | nd | |
| 25965 | 82A | 85% | (0.7 mg/g) |
| 25966 | 81B, 82A | 83% | (0.4 mg/g) |
| 25973 | 82b | 84% | (0.5 mg/g) |
| 25976 | 81B | 84% | (0.3 mg/g) |
| FLORIGENE Moondust | 76A | 100% | (0.035 mg/g) |
| FLORIGENE Moonshadow | 82A, 82B | 94% | (0.35 mg/g) |
| FLORIGENE Moonshade | 81A, 82A | 97% | (0.6 mg/g) |
| FLORIGENE Moonlite | 77D/82D, 77C | 71% | (0.06 mg/g) |
| FLORIGENE Moonaqua | 84A/B | 74% | (0.07 mg/g) |
| FLORIGENE Moonvista | 81A+ | 98% | (1.8 mg/g) |

Accession number = unique number given to individual transgenic event
RHSCC number = The color code of the petal limbs from the flowers of transgenic carnation lines. “+” alongside an RHSCC number highlights that the color is a darker or more intense shade of the selected code
delphinidin levels = delphinidin levels detected in hydrolyzed extracts of petal limb tissue as determined by HPLC given in percentage of total anthocyanidins and mg/g of fresh weight of petal tissue.
nd = not done

Further field trial assessments in Colombia revealed that lines #25958, #25947, #25973, #25965 and #25976 produced novel spray carnation flower colors with consistent and stable colors and good plant growth characteristics. Two lines (#25958 and #25947) were selected for commercialization. Line #25958 was subsequently named FLORIGENE Moonberry (Trade mark) and line #25947 was called FLORIGENE Moonpearl (Trade mark). Both are being grown in Colombia for production of cut flowers to markets around the world.

Introduction of the Transformation Vector pCGP3366 into Other Carnation Varieties

Due to the success in obtaining high delphinidin levels in the carnation variety, Cerise

Westpearl using the construct pCGP3366 (containing at least one F3′5′H enzyme and at least one DFR enzyme and incorporation of at least one ds carnDFR molecule) the same genes are introduced into other colored carnation cultivars such as but not limited to Cinderella, Westpearl, Vega, Artisan, Barbara, Dark Rendezvous, Miledy, Kortina Chanel.

The transgenic plants are assessed for flower color as described above and lines with novel flower color (as compared to controls) are selected for commercialization.

Use of the Binary Vector pCGP3366 as a Backbone-Addition of Other Expression Cassettes.

In order to shift petal color further towards the blue/purple spectrum other genes that modulated anthocyanin or flavonoid composition were added to the pCGP3366 binary vector. These included genes coding for S-adenosylmethionine: anthocyanin 3′5′ methyltransferase (AMT) activity to modulate the production of methylated anthocyanins such as the production of malvidin and petunidin pigments and genes coding for flavone synthase (FNS) activity to modulate the production of flavones in carnation.

Addition of AMT Expression Cassettes to the pCGP3366 Binary Construct

In an attempt to produce anthocyanins based upon malvidin (the methylated form of delphinidin) 2 new transformation vectors, pCGP3601 and pCGP3605, were prepared by addition of AMT expression cassettes to the transformation vector, pCGP3366 (FIG. 3). The AMT sequence from torenia (International Patent Application No. PCT/AU03/00079) was used under the control of a floral specific promoter fragment from the ANS gene of carnation (carnANS 5′) and a constitutive promoter fragment from the cauliflower mosaic virus 35S gene (CaMV35S).

The Transformation Vector, pCGP3601 (carnANS 5′:ThMT:carnANS 3; AmCHS 5′: BPF3′5′H#40:petD8 3′; Pet gen DFR; CaMV35S 5′:ds carn DFR:35S 3; 35S 5′: SuRB)

The binary construct pCGP3601 contains a carnANS 5′:ThMT:carnANS 3′ expression cassette in the pCGP3366 binary construct backbone (described above) (FIG. 3).

Construction of the Intermediate Plasmid, pCGP3431 (carnANS 5′:ThMT:carnANS 3)

A ˜1.0 kb fragment bearing the torenia AMT cDNA clone (ThMT) (SEQ ID NO: 11) was released from the plasmid pTMT5 (described in International Patent Application No.: PCT/JP00/00490) upon digestion with the restriction endonucleases EcoRI and Asp718. The overhanging ends were repaired and the purified fragment was ligated with XbaI/PstI repaired ends of the plasmid pCGP1275 (described in International Patent Application No. PCT/AU2008/001700 incorporated herein by reference). Correct insertion of the ThMT fragment in between a promoter fragment of the carnation ANS gene (carnANS 5) and a terminator fragment of the carnation ANS gene (carnANS 3) was established by restriction endonuclease analysis of plasmid DNA isolated from ampicillin resistant transformants. The resulting plasmid was designated as pCGP3431.

Construction of the Transformation Vector, pCGP3601

A 4.4 kb fragment harboring the carnANS 5′:ThMT:carnANS 3′ expression cassette was isolated from the plasmid pCGP3431 (described above) upon digestion with the restriction endonuclease ClaI. The overhanging ends were repaired and the purified fragment was ligated with the PmeI ends of the plasmid pCGP3366 (described above) (FIG. 3). Correct insertion of the carnANS 5′:ThMT:carnANS 3′ expression cassette in a tandem orientation with respect to the AmCHS 5′:BPF3′5′H#40:petD8 3, pet gen DFR; CaMV35S 5′:ds carn DFR:35S 3′ and 35S 5′:SuRB genes was established by restriction endonuclease analysis of plasmid DNA isolated from tetracycline-resistant transformants. The resulting plasmid was designated as pCGP3601 (FIG. 4).

The Transformation Vector, pCGP3605 (CaMV35S:ds cam DFR:35S 3; CaMV35S: ThMT:35S 3; Pet gen DFR; AmCHS 5′:BPF3′5′H#40:petD8 3; 35S 5′:SuRB)

The binary construct pCGP3605 contains a CaMV35S:ThMT:35S 3′ expression cassette in the pCGP3366 binary construct backbone (described above) (FIG. 3).

Construction of the Intermediate Plasmid, pCGP3097 (CaMV35S:ThMT:35S 3)

The plasmid pTMT5 (described in International Patent Application No. PCT/JP00/00490) was firstly linearized upon digestion with the restriction endonuclease Asp718. The overhanging ends were repaired and a ˜1.0 kb fragment bearing the torenia AMT cDNA clone (ThMT) (SEQ ID NO: 11) was then released from the linearized plasmid upon digestion with the restriction endonuclease EcoRI. The fragment was purified and ligated with XbaI (repaired ends)/EcoRI ends of the plasmid pRTppoptcAFP (a source of the CaMV35S promoter and terminator fragments) (Wnendt et al., 1994, supra). Correct insertion of the ThMT fragment in a sense orientation between the promoter and terminator fragments of the cauliflower mosaic virus 35S gene (CaMV35S and 35S 3′ respectively) was established by restriction endonuclease analysis of plasmid DNA isolated from ampicillin resistant transformants. The resulting plasmid was designated as pCGP3097.

Construction of the Transformation Vector, pCGP3605

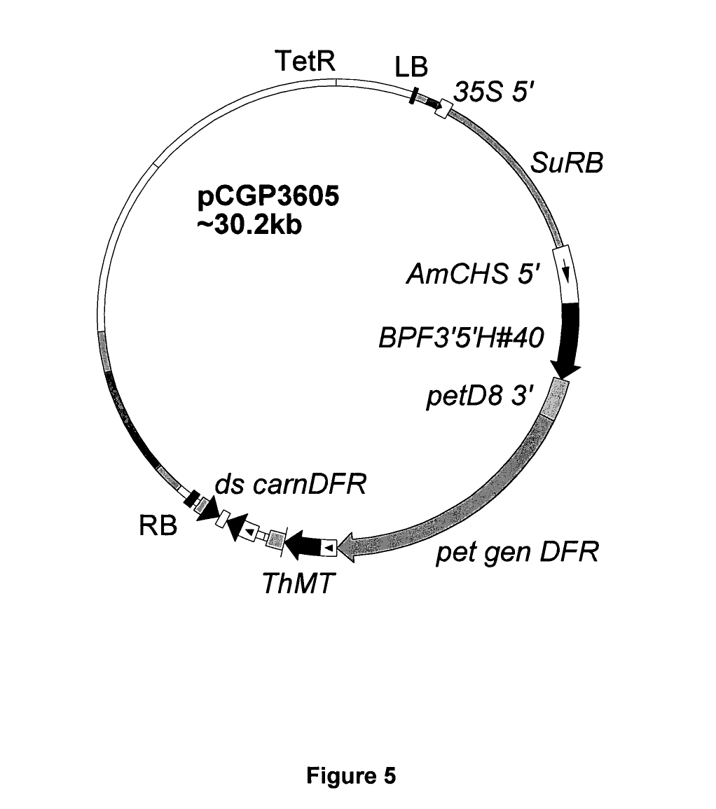

A ˜1.6 kb fragment harboring the CaMV35S:ThMT: 35S 3′ expression cassette was isolated from the plasmid pCGP3097 (described above) upon digestion with the restriction endonuclease PstI. The overhanging ends were repaired and the purified fragment was ligated with the PmeI ends of the plasmid pCGP3366 (described above) (FIG. 3). Correct insertion of the CaMV35S:ThMT:35S 3′ expression cassette in a tandem orientation with respect to the AmCHS 5′:BPF3′5′H#40:petD8 3, pet gen DFR; CaMV35S:ds carn DFR:35S 3′ and 35S 5′:SuRB genes was established by restriction endonuclease analysis of plasmid DNA isolated from tetracycline-resistant transformants. The resulting plasmid was designated as pCGP3605 (FIG. 5).

Addition of FNS Expression Cassettes to the pCGP3366 Binary Construct

In an attempt to produce flavones (to act as co-pigments) and high levels of delphinidin in a Cerise Westpearl background, a further 2 transformation vectors, pCGP3616 and pCGP3607 were prepared by adding FNS expression cassettes to the transformation vector, pCGP3366 (FIG. 3). The FNS sequence from torenia (International Patent Application No. PCT/JP00/00490) (SEQ ID NO: 13) was used under the control of a floral specific promoter fragment from the CHS gene of rose (RoseCHS 5) and a constitutive promoter fragment from the cauliflower mosaic virus 35S gene (CaMV35S).

The Transformation Vector, pCGP3616 (CaMV35S:ds carn DFR:35S 3; RoseCHS 5′: ThFNS:nos 3; Pet gen DFR; AmCHS 5′:BPF3′5′H#40:petD8 3; 35S 5′:SuRB)

The binary construct pCGP3616 contains a RoseCHS 5′:ThFNS:nos 3′ expression cassette in the pCGP3366 binary construct backbone (described above) (FIG. 3).

Construction of the Intermediate Plasmid, pCGP3123 (RoseCHS 5′:ThFNS:nos 3)

A 3.2 kb fragment bearing e35S 5′:ThFNS:petD8 3′ expression cassette was released from the binary vector plasmid pSFL535 (described in International Patent Application WO2008/156206) upon digestion with the restriction endonuclease AscI. The fragment was purified and ligated with the AscI ends of the 2.9 kb plasmid pUCAP+AscI (The plasmid pUCAP/AscI is a pUC19 based cloning vector with extra cloning sites specifically an AscI recognition site at either ends of the multicloning site). Correct insertion of the e35S 5′: ThFNS:petD8 3′ expression cassette in the pUC based cloning vector was established by restriction endonuclease analysis of plasmid DNA isolated from ampicillin resistant transformants. The resulting plasmid was designated as pCGP3123.

Construction of the Intermediate Plasmid, pCGP3612 (RoseCHS 5′:ThFNS:nos 3)

This plasmid pCGP3123 (described above) was linearized upon digestion with the restriction endonuclease BamHI. The overhanging ends were repaired and a fragment bearing the ThFNS cDNA clone was then released after partial digestion of the linearized plasmid with the restriction endonuclease XhoI. The 1.7 kb fragment was purified and ligated with SmaI/XhoI ends of the plasmid pCGP2203 (Rose CHS 5′:BPF3′5′H#18:nos 3′ in pBluescript backbone) described in International Patent Application No. PCT/AU2008/001694. Correct insertion of the ThFNS fragment between the RoseCHS promoter and nos terminator was established by restriction endonuclease analysis of plasmid DNA isolated from ampicillin resistant transformants. The resulting plasmid was designated pCGP3612.

Construction of the Transformation Vector, pCGP3616

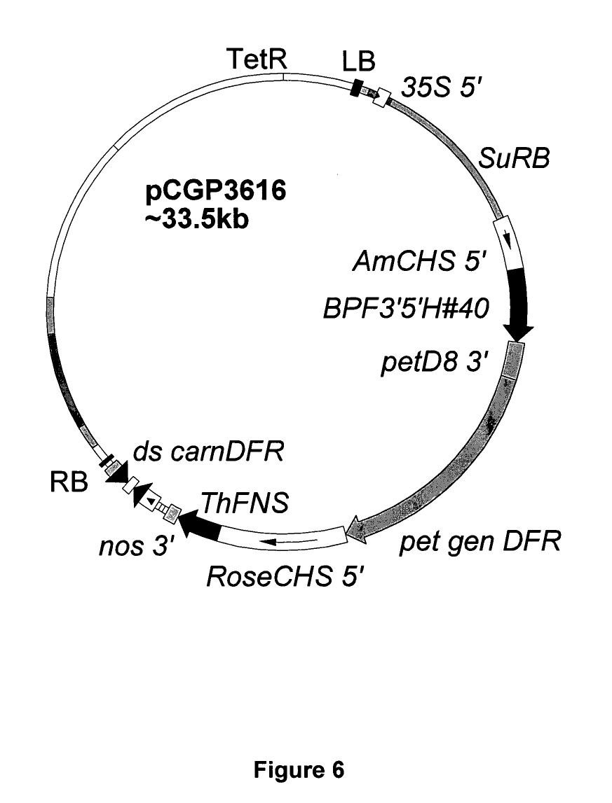

A 4.9 kb fragment harboring the RoseCHS 5′:ThFNS:nos 3′ expression cassette was isolated from the plasmid pCGP3612 (described above) upon digestion with the restriction endonucleases BglII and NotI. The overhanging ends were repaired and the purified fragment was ligated with the PmeI ends of the plasmid pCGP3366 (described above) (FIG. 3). Correct insertion of the RoseCHS 5′:ThFNS:nos 3′ expression cassette in a tandem orientation with respect to the AmCHS 5′:BPF3′5′H#40:petD8 3, pet gen DFR; CaMV35S:ds carn DFR:35S 3′ and 35S 5′:SuRB genes was established by restriction endonuclease analysis of plasmid DNA isolated from tetracycline-resistant transformants. The resulting plasmid was designated as pCGP3616 (FIG. 6).

The Transformation Vector, pCGP3607 (CaMV35S′:ds carn DFR:35S 3; e35S 5′: ThFNS:petD8 3; Pet gen DFR; AmCHS 5′:BPF3′5′H#40:petD8 3; 35S 5′:SuRB)

The binary construct pCGP3607 contains an e35S 5′:ThFNS:petD8 3′ expression cassette in the pCGP3366 binary construct backbone (described above) (FIG. 3).

Construction of the Transformation Vector, pCGP3607

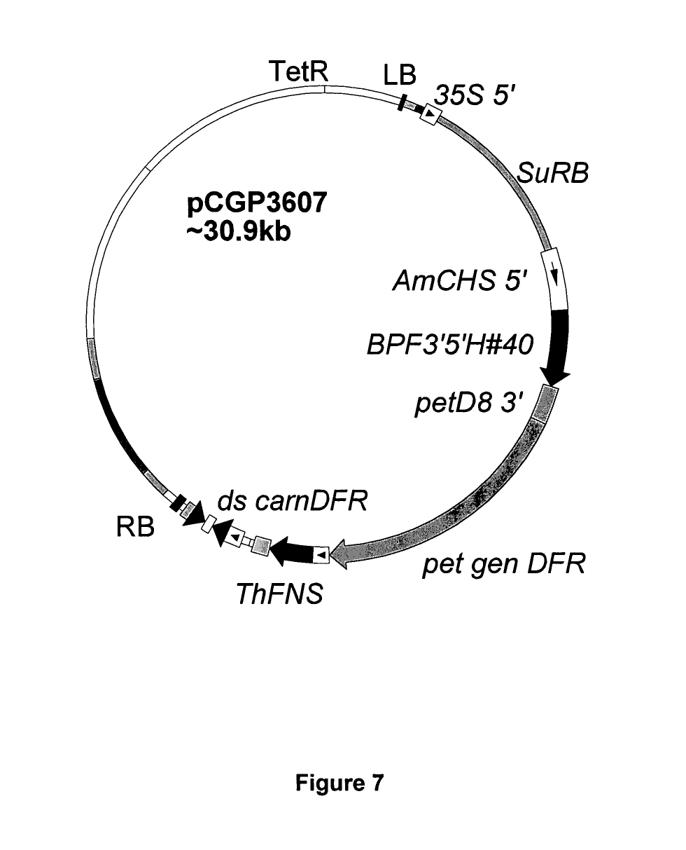

A 3.2 kb fragment bearing e35S 5′:ThFNS:petD8 3′ expression cassette was released from the plasmid pCGP3123 (described above) upon digestion with the restriction endonuclease AscI. The fragment was purified and ligated with the PmeI ends of the plasmid pCGP3366 (described above) (FIG. 3). Correct insertion of the e35S 5′:ThFNS:petD8 3′ expression cassette in a tandem orientation with respect to the AmCHS 5′:BPF3′5′H#40: petD8 3, pet gen DFR; CaMV35S:ds carn DFR:35S 3′ and 35S 5′:SuRB genes was established by restriction endonuclease analysis of plasmid DNA isolated from tetracycline-resistant transformants. The resulting plasmid was designated as pCGP3607 (FIG. 7).

The T-DNAs of the transformation vectors pCGP3601 (FIG. 4), pCGP3605 (FIG. 5), pCGP3607 (FIG. 7) and pCGP3616 (FIG. 6) were introduced into the spray carnation line, Cerise Westpearl via Agrobacterium-mediated transformation. Transgenic cells were selected based on their ability to grow and produce roots on media containing the herbicide, chlorsulfuron. Transgenic plantlets with roots were removed form media and transferred to soil and grown to flowering in temperature controlled greenhouses in Bundoora, Victoria, Australia. The results are summarized in Table 6.

TABLE 6
A summary of the number of transgenic Cerise Westpearl that resulted
in a significant shift in petal color towards the purple/violet range.

| Construct | Addition to pCGP3366 | #Tg | CC |
|---|---|---|---|
| pCGP3601 | carnANS 5′: ThMT: carnANS 3′ | 32 | 11 |
| pCGP3605 | CaMV35S: ThMT: 35S 3′ | 38 | 14 |
| pCGP3607 | e35S 5′: ThFNS: petD8 3′ | 37 | 15 |
| pCGP3616 | RoseCHS 5′: ThFNS: nos 3′ | 19 | 2 |

Construct = plasmid pCGP identification number of the transformation vector used in the transformation experiment
Addition to pCGP3366 = Extra expression cassette added to the pCGP3366 (FIG. 3) backbone containing AmCHS 5′: BP F3′5′H #40: petD8 3′; Pet gen DFR; CaMV 35S: ds carnDFR: 35S 3′ transgenes
#Tg = total number of transgenic carnation lines produced
CC = “Color Change” -the number of events produced that had a shift in petal color towards the purple range

The transgenic plants are assessed for flower color as described above and lines with novel flower color (as compared to controls) are selected for commercialization.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Altschul et al, J. Mol. Biol. 215(3):403-410, 1990
Barker et al, Plant Mol. Biol. 2:235-350, 1983
Beld et al, Plant Mol. Biol. 13:491-502, 1989
Bonner and Laskey, Eur. J. Biochem. 46:83, 1974
Brouillard and Dangles, In: The Flavonoids—Advances in Research since 1986, Harborne, (ed), Chapman and Hall, London, UK, 1-22, 1993
Brugliera et al, Plant J. 5:81-92, 1994
Bullock et al, Biotechniques 5:376, 1987
Forkmann and Ruhnau, Naturforsch C. 42c, 1146-1148, 1987
Franck et al, Cell 21:285-294, 1980
Fukui et al, Phytochemistry.63(1), 15-23, 2003
Garfinkel et al, Cell 27:143-153, 1983
Glimn-Lacy and Kaufman, Botany Illustrated, Introduction to Plants, Major Groups, Flowering Plant Families, 2′ ed, Springer, USA, 2006
Greve, J. Mol. Appl. Genet. 1:499-511, 1983
Guilley et al, Cell 30:763-773, 1982
Harpster et al, MGG 212:182-190, 1988
Holton, Isolation and characterization of petal-specific genes from Petunia hybrida. PhD Thesis, University of Melbourne, 1992
Holton et al, Nature, 366:276-279, 1993
Holton and Cornish, Plant Cell 7:1071-1083, 1995
Huang and Miller, Adv. Appl. Math. 12:373-381, 1991
Inoue et al, Gene 96:23-28, 1990
Katsumoto et al, Plant Cell Physiol. 48(11), 1589-1600, 2007
Johnson et al Plant Journal, 19:81-85, 1999
Lazo et al, Bio/technology 9:963-967, 1991
Lee et al, EMBOJ 7:1241-1248, 1988
Lu et al, Bio/Technology 9:864-868, 1991
Marmur and Doty, J. Mol. Biol. 5:109, 1962
Mitsuhashi et al. Plant Cell Physiol. 37: 49-59, 1996
Mol et al, Trends Plant Sci. 3:212-217, 1998
Nakayama et al, Phytochemistry, 55, 937-939, 2000
Ossowski et al, Plant J, 53, 674-690, 2008
Pearson and Lipman, Proc. Natl. Acad. Sci. USA 85(8):2444-2448, 1988
Plant Molecular Biology Labfax, Croy (ed), Bios scientific Publishers, Oxford, UK, 1993
Plant Molecular Biology Manual (2^{nd} edition), Gelvin and Schilperoot (eds), Kluwer Academic Publisher, The Netherlands, 1994
Salomon et al, EMBO, J. 3:141-146, 1984
Sambrook et al, Molecular Cloning: A Laboratory Manual, Cold Spring Harbor Laboratories, Cold Spring Harbor, N.Y., USA, 1989
Sambrook and Russell, Molecular Cloning: A Laboratory Manual 3^{rd} edition, Cold Spring Harbor Laboratories, Cold Spring Harbor, N.Y., USA, 2001
Seitz and Hinderer, Anthocyanins. In: Cell Culture and Somatic Cell Genetics of Plants. Constabel and Vasil (eds.), Academic Press, New York, USA, 5:49-76, 1988
Sommer and Saedler, Mol. Gen. Genet 202:429-434, 1986
Strack and Wray, In: The Flavonoids—Advances in Research since 1986. Harborne, J. B. (ed), Chapman and Hall, London, UK, 1-22, 1993
Tanaka and Mason, In Plant Genetic Engineering, Singh and Jaiwal (eds) SciTech Publishing Llc., USA, 1:361-385, 2003,
Tanaka et al, Plant Cell, Tissue and Organ Culture 80:1-24, 2005
Tanaka and Brugliera, In Flowering and Its Manipulation, Annual Plant Reviews Ainsworth (ed), Blackwell Publishing, UK, 20:201-239, 2006)
Thompson et al, Nucleic Acids Research 22:4673-4680, 1994
Wnendt et al., Curr Genet 25: 510-523, 1994
Wesley et al, Plant J., 27, 581-590, 2001
Winkel-Shirley, Plant Physiol. 126:485-493, 2001a
Winkel-Shirley, Plant Physiol. 127:1399-1404, 2001b

1. A genetically modified plant exhibiting altered inflorescence, said plant or its progeny comprising expressed genetic material encoding at least one non-indigenous flavonoid 3′,5′ hydroxylase (F3′5′H) enzyme and at least one non-indigenous dihydroflavonol 4-reductase (DFR) enzyme and expressing genetic material which down regulates expression of the plant's indigenous DFR gene.

2. The genetically modified plant wherein the plant or its progeny further comprise expressed genetic material encoding a non-indigenous S-adenosylmethionine: anthocyanin 3′5′ methyltransferase (ThMT) and/or flavone synthase (ThFNS).

3. The genetically modified plant of claim 1 wherein the genetic material which down regulates the indigenous DFR gene is sense and anti-sense nucleotide sequences corresponding to the plant's indigenous DFR gene (ds plantDFR).

4. The genetically modified plant of claim 3 wherein the plant is a carnation and the ds plantDFR is ds carnDFR.

5. The genetically modified plant of claim 4 wherein the carnation is in a Cerise Westpearl background.

6. The genetically modified plant of claim 1 wherein the altered inflorescence is a color in the range of purple to violet mauve.

7. The genetically modified plant of claim 6 wherein the altered inflorescence is mauve.

8. The genetically modified plant of claim 5 wherein the carnation is in the spray carnation Dianthus caryophyllus cv. Cerise Westpearl genetic background or a sport thereof.

9. The genetically modified plant of claim 5 wherein the carnation has the genetic background of Vega, Artisan, Cinderella, Westpearl, Barbara, Miledy, Dark Rendezvous, Kortina Chanel.

10. The genetically modified plant of claim 1 wherein the F3′5′H enzyme is from Viola sp.

11. The genetically modified plant of claim 10 wherein the F3′5′H enzyme is encoded by SEQ ID NO:1, or a nucleotide sequence capable of hybridizing to a complementary form of SEQ ID NO:1 under medium stringency conditions.

12. The genetically modified plant of claim 10 wherein the F3′5′H enzyme is encoded by SEQ ID NO:1.

13. The genetically modified plant of claim 1 wherein the DFR is from Petunia sp.

14. The genetically modified plant of claim 13 wherein the DFR is encoded by SEQ ID NO:3 or a nucleotide sequence capable of hybridizing to a complementary form of SEQ ID NO:3 under medium stringency conditions.

15. The genetically modified plant of claim 14 wherein the DFR is encoded by SEQ ID NO:3.

16. The genetically modified plant of claim 3 wherein the ds plantDFR is from Dianthus sp (ds dianDFR).

17. The genetically modified plant of claim 16 wherein the ds plantDFR incorporates a fragment or fragments from by SEQ ID NO:9 or a nucleotide sequence capable of hybridizing to a complementary form of SEQ ID NO:9 under high stringency conditions.

18. The genetically modified plant of claim 17 wherein the ds plantDFR incorporates a fragment or fragments from by SEQ ID NO:9.

19. The genetically modified plant of claim 5 wherein the plant is Cerise Westpearl/3366.

20. Progeny, reproductive material, cut flowers, tissue culturable cells and regenerable cells from the genetically modified plant of claim 1.

21. (canceled)

22. The method of claim 25, further comprising introducing genetic material encoding a non-indigenous ThMT and/or ThFNS.

23. The method of claim 22 wherein carnation plant is a Cerise Westpearl carnation.

24. The method of claim 23 wherein the genetic material down regulates expression of the plant's indigenous DFR gene comprises sense and anti-sense nucleotide sequences corresponding to the plant's indigenous DFR gene.

25. A method for producing a carnation exhibiting altered inflorescence, said method comprising introducing into regenerable cells of a carnation plant expressible genetic material encoding at least one non-indigenous F3′5′H enzyme and at least one non-indigenous DFR enzyme and incorporation of genetic material which down regulates expression of a plant's indigenous DFR gene and regenerating a plant therefrom or obtaining progeny of the regenerated plant.

26. A method for producing a carnation line exhibiting altered inflorescence, the method comprising selecting a spray carnation comprising genetic material encoding one of at least one non-indigenous F3′5′H enzyme or at least one non-indigenous DFR enzyme or incorporation of at least one ds carnDFR molecule and crossing this plant with another carnation comprising genetic material encoding the other of at least one non-indigenous F3′5′H enzyme or at least one non-indigenous DFR enzyme or incorporation of at least one ds carnDFR molecule and then selecting F1 or subsequent generation plants which express the genetic material.

27. A method for producing a carnation line exhibiting altered inflorescence, said method comprising selecting a spray carnation comprising genetic material encoding one of at least one non-indigenous F3′5′H enzyme or at least one non-indigenous DFR enzyme or incorporation of at least one ds carnDFR molecule and crossing this plant with another carnation and then selecting F1 or subsequent generation plants which express the genetic material.
